SEQUENCE LISTING (10 sequences)

SEQ ID NO: 1 - DNA, 3718 bp, Homo sapiens
gtggtctcgcgggcgggtgacgtcatcgaggcgagagaggcggagccgtggacagtgggcgggaggctgccaacggttttgatcctagggggaggcgtgagagggggatctcaggggaggaggtcaatcgcttgccccccactttggcaaattggggactgaggactggaagggtggagagtaggcggaaccaggtggtcgtcggggcagaggatctcgggctaggcttgagggcggcgtgcttcttagggacgacttagggcgtgactgagggttcacaaggtttcttttggggtggtcgggagggagagattctagggaacaaggaagctcgctatggctttcttgccaggaggggtcgaagggaaagtacaagggagctgaccctgggtagaacgggtgaagggatgggggagcgtgaggttccgccctctcttgagactggaaccaattgagggactagtagggcagggggacagaaattgggctcctagtggatttgggtccgtttccgttgggacgttttgggtgtgagaacttaagagctcagttgaccggggatagcctgtgccggagttgatctgcagcttccagcactcgtagtcgggaagaggagcttcagcagcgctgttgtcccacagtagggcttctgtccgcacccgctctgcgctgcaccctcttaacgctgttcccaggagctggggaaagggatgcttttgcccactcccatggcccctggaactggtggaaacctttcctctaaccagaaagcctcgatatccttaattcaccaaggatccttggcgtggagtcttcctcccttctcccaagtctttctccgtgaacttttcctcctggactttgctaaagcagaacctcccagctctttgctgtctccggttgtctcttccctttattcatggcaacatcagctgacagccccagttcacccctcggggcggaggatctcctgagtgattcatcagaaccccctgggctcaaccaagtgtcgtctgaagtgacctcccagctctatgcttctttgcgcctcagccggcaggcggaggccacggcccgagcccagctgtatttaccctccacctccccgcctcatgaagggttagacggcttcgcccaagaattgagtcgaagcttgtcagtcggattggaaaagaacttgaagaaaaaggatggttctaagcatatctttgagatggaaagtgttcggggtcagctccagaccatgctccaaacctcacgtgatacagcctatcgggatcctctcattcctggcgctggctcagagagacgggaagaggactcctttgacagtgatagcacagccaccttgctcaacacccggcccctgcaagacttgtctccatctagctcagcccaagccctggaggagctgtttccccgctacaccagccttcggccagggcctccactcaatcccccagattttcaggggctgagagatgcattggattcagagcatacccgccgcaagcattgtgagcgccatattcagagcctgcagacccgagtgttagagctacagcaacaattagccgtggctgtggctgccgaccgcaagaaagataccatgattgaacaactggacaagaccctggcccgtgtggtggagggctggaaccggcatgaggctgagcggacagaggttctcaggggacttcaagaggaacaccaggcagcagagctcaccagaagcaagcagcaggagacagtaacccgcctggaacaaagcctttctgaggccatggaggccctgaatcgtgagcaggaaagtgccagactgcagcaacgggaaagagagacaatggaggaggaaaggcaagctctgactctgaggttggaggcagaacagcagcggtgctgtgtcctgcagaaagagcgggatgcagctcgggctgggcaactgagtgagcatcgagagttggagactcttcgggctgccctagaagaagaacggcagacctgggcccagcaagagcaccagcttaaggaacactaccaggcgctgcaggaggagagccaggctcagctggaaagagagaaggagaagagccagagggaagcccaggccgcctgggagacccagcaccagttggcattggtgcagtctgaggtgcggcggctggaaggagagctggatacagctcggagagagagagatgccctgcagctggaaatgagcttggtgcaggcccggtatgaaagccagcggatccagctggagtcggagctggctgtgcagctggagcagcgggtgacagagcggctggcgcaggctcaggaaagcagcctacggcaagcagcctccctcagggaacatcacaggaagcagctgcaggacctgagtggacagcaccagcaggagctggccagtcagctagctcagttcaaggtggaaatggcagaacgagaggaacggcaacagcaggtggctgaggactacgagctcagactggcccgggagcaagcgcgagtgtgcgaactgcagagtgggaaccagcagctggaggagcagcgggtggagctggttgaaagactgcaggccatgctgcaggcccactgggatgaggccaaccagctgctcagcaccactctcccgccgcccaaccctccagctcctcctgctggaccctccagccccgggcctcaggagcccgagaaggaggaaaggagggtctggactatgcctcccatggccgtggccctgaagcctgtattgcagcagagccgggaagcaagggacgagctacctggagcgcctcctgttctttgcagttcctcctcagatcttagcctcctgttgggcccctcttttcagagccagcattctttccagcccctggagcccaaaccagacctcacttcatccacagctggggccttctctgcacttggggccttccatcccgatcatagggcagaaaggccattccctgaggaagatcctggacctgacggggagggcctcctaaagcaagggctgccgcctgctcagctggagggcctcaagaattttttgcaccagttgctggagacagtgccccagaacaatgagaacccttctgtcgacctgttgccccctaagtctggtcctctgactgtcccatcttgggaggaagcccctcaagtgccacgtattccaccgcctgtccacaaaaccaaagttcccttagccatggcatccagtcttttccgggtccctgagcctctctcctcccattcacaaggcagtggtcccagcagtggttccccagagagaggtggagatgggcttacattcccaaggcagctgatggaggtgtctcaactgttgcgactctaccaggctcggggctggggggctctgcctgctgaggatctcctgctctacctgaagaggctggaacacagcgggactgatggccgaggggataatgtccccagaaggaacacagactcccgcttgggtgagatcccccggaaagagattccctcccaggctgtccctcgccgccttgctacagcccccaagactgaaaaacctcccgcacggaagaaaagtgggcaccctgccccgagtagcatgaggagccgggggggagtctggagatgagcccccctaccctctctcctctttgttctctcattgttgttattttaataaatgctcattagtctgcaaaaaaaaaaaaaaaaaaaa

SEQ ID NO: 2 - DNA, 2712 bp, Homo sapiens
atggcaacatcagctgacagccccagttcacccctcggggcggaggatctcctgagtgattcatcagaaccccctgggctcaaccaagtgtcgtctgaagtgacctcccagctctatgcttctttgcgcctcagccggcaggcggaggccacggcccgagcccagctgtatttaccctccacctccccgcctcatgaagggttagacggcttcgcccaagaattgagtcgaagcttgtcagtcggattggaaaagaacttgaagaaaaaggatggttctaagcatatctttgagatggaaagtgttcggggtcagctccagaccatgctccaaacctcacgtgatacagcctatcgggatcctctcattcctggcgctggctcagagagacgggaagaggactcctttgacagtgatagcacagccaccttgctcaacacccggcccctgcaagacttgtctccatctagctcagcccaagccctggaggagctgtttccccgctacaccagccttcggccagggcctccactcaatcccccagattttcaggggctgagagatgcattggattcagagcatacccgccgcaagcattgtgagcgccatattcagagcctgcagacccgagtgttagagctacagcaacaattagccgtggctgtggctgccgaccgcaagaaagataccatgattgaacaactggacaagaccctggcccgtgtggtggagggctggaaccggcatgaggctgagcggacagaggttctcaggggacttcaagaggaacaccaggcagcagagctcaccagaagcaagcagcaggagacagtaacccgcctggaacaaagcctttctgaggccatggaggccctgaatcgtgagcaggaaagtgccagactgcagcaacgggaaagagagacaatggaggaggaaaggcaagctctgactctgaggttggaggcagaacagcagcggtgctgtgtcctgcagaaagagcgggatgcagctcgggctgggcaactgagtgagcatcgagagttggagactcttcgggctgccctagaagaagaacggcagacctgggcccagcaagagcaccagcttaaggaacactaccaggcgctgcaggaggagagccaggctcagctggaaagagagaaggagaagagccagagggaagcccaggccgcctgggagacccagcaccagttggcattggtgcagtctgaggtgcggcggctggaaggagagctggatacagctcggagagagagagatgccctgcagctggaaatgagcttggtgcaggcccggtatgaaagccagcggatccagctggagtcggagctggctgtgcagctggagcagcgggtgacagagcggctggcgcaggctcaggaaagcagcctacggcaagcagcctccctcagggaacatcacaggaagcagctgcaggacctgagtggacagcaccagcaggagctggccagtcagctagctcagttcaaggtggaaatggcagaacgagaggaacggcaacagcaggtggctgaggactacgagctcagactggcccgggagcaagcgcgagtgtgcgaactgcagagtgggaaccagcagctggaggagcagcgggtggagctggttgaaagactgcaggccatgctgcaggcccactgggatgaggccaaccagctgctcagcaccactctcccgccgcccaaccctccagctcctcctgctggaccctccagccccgggcctcaggagcccgagaaggaggaaaggagggtctggactatgcctcccatggccgtggccctgaagcctgtattgcagcagagccgggaagcaagggacgagctacctggagcgcctcctgttctttgcagttcctcctcagatcttagcctcctgttgggcccctcttttcagagccagcattctttccagcccctggagcccaaaccagacctcacttcatccacagctggggccttctctgcacttggggccttccatcccgatcatagggcagaaaggccattccctgaggaagatcctggacctgacggggagggcctcctaaagcaagggctgccgcctgctcagctggagggcctcaagaattttttgcaccagttgctggagacagtgccccagaacaatgagaacccttctgtcgacctgttgccccctaagtctggtcctctgactgtcccatcttgggaggaagcccctcaagtgccacgtattccaccgcctgtccacaaaaccaaagttcccttagccatggcatccagtcttttccgggtccctgagcctctctcctcccattcacaaggcagtggtcccagcagtggttccccagagagaggtggagatgggcttacattcccaaggcagctgatggaggtgtctcaactgttgcgactctaccaggctcggggctggggggctctgcctgctgaggatctcctgctctacctgaagaggctggaacacagcgggactgatggccgaggggataatgtccccagaaggaacacagactcccgcttgggtgagatcccccggaaagagattccctcccaggctgtccctcgccgccttgctacagcccccaagactgaaaaacctcccgcacggaagaaaagtgggcaccctgccccgagtagcatgaggagccgggggggagtctggagatga

SEQ ID NO: 3 - protein, 903 aa, Homo sapiens
MATSADSPSSPLGAEDLLSDSSEPPGLNQVSSEVTSQLYASLRLSRQAEATARAQLYLPSTSPPHEGLDGFAQELSRSLSVGLEKNLKKKDGSKHIFEMESVRGQLQTMLQTSRDTAYRDPLIPGAGSERREEDSFDSDSTATLLNTRPLQDLSPSSSAQALEELFPRYTSLRPGPPLNPPDFQGLRDALDSEHTRRKHCERHIQSLQTRVLELQQQLAVAVAADRKKDTMIEQLDKTLARVVEGWNRHEAERTEVLRGLQEEHQAAELTESKQQETVTRLEQSLSEAMEALNREQESARLQQRERETLEEERQALTLRLEAEQQRCCVLQEERDAARAGQLSEHRELETLRAALEEERQTWAQQEHQLKEHYQALQEESQAQLEREKEKSQREAQAAWETQHQLALVQSEVRRLEGELDTARRERDALQLEMSLVQARYESQRIQLESELAVQLEQRVTERLAQAQESSLRQAASLREHHRKQLQDLSGQHQQELASQLAQFKVEMAEREERQQQVAEDYELRLAREQARVCELQSGNQQLEEQRVELVERLQAMLQAHWDEANQLLSTTLPPPNPPAPPAGPSSPGPQEPEKEERRVWTMPPMAVALKPVLQQSREARDELPGAPPVLCSSSSDLSLLLGPSFQSQHSFQPLEPKPDLTSSTAGAFSALGAFHPDHRAERPFPEEDPGPDGEGLLKQGLPPAQLEGLKNFLHQLLETVPQNNENPSVDLLPPKSGPLTVPSWEEAPQVPRIPPPVHKTKVPLAMASSLFRVPEPPSSHSQGSGPSSGSPERGGDGLTFPRQLMEVSQLLRLYQARGWGALPAEDLLLYLKRLEHSGTDGRGDNVPRRNTDSRLGEIPRKEIPSQAVPRRLATAPKTEKPPARKKSGHPAPSSMRSRGGVWR

SEQ ID NO: 4 - DNA, 3784 bp, Homo sapiens
gtggtctcgcgggcgggtgacgtcatcgaggcgagagaggcggagccgtggacagtgggcgggaggctgccaacggttttgagcgtagggggaggcgtgagagggggatctcaggggaggaggtcaatcgcttgccccccactttggcaaattggggactgaggactggaaggatggagagtaggcggaaccaggtggtcgtcggggcagaggatctcgggctaggcttgagggcggcgtgcttcttagggacgacttagggcgtgactgagggttcacaaggtttcttttggggtggtcgggagggagagattctagggaacaaggaagctcgctatggctttcttgccaggaggggtcgaagggaaagtacaagggagctgaccctgggtagaacgggtgaagggatgggggagcgtgaggttccgccctctcttgagactggaaccaattgagggactagtagggcagggggacagaaattgggctcctagtggatttgggtccgtttccgttgggacgttttgggtgtgagaacttaagagctcagttgaccggggatagcctgtgccggagttgatctgcagcttccagcactcgtagtcgggaagaggagcttcagcagcgctgttgtcccacagtaggtcttctgtccgcacccgctctgcgctgcaccctcttaacgctgttcccaggagctgggaaaagggatgcttttgcccactcccatggcccctggaactggtggaaacctttcctctaaccagaaagcctcgatatccttaattcaccaaggatccttggcgtggagtcttcctcccttctcccaagtctttctccgtgaacttttcctcctggactttgctaaagcagaacctcccagctctttgctgtctccggttgtctcttccctgtattcatggcaacatcagctgacagccccagttcacccctcgggtcggaggatctcctgagtgattcatcagaaccccctgggctcaaccaagtgtcgtctgaagtgacctcccagctctatgcttctttgcgcctcagccggcaggcggaggccacggcccgagcccagctgtatttaccctccacctccccgcctcatgaagggttagacggcttcgcccaagaattgagtcgaagcttgtcagtcggattggaaaagaacttgaagaaaaaggatggttctaagcatatctttgagatggaaagtgttcggggtcagctccagaccatgctccaaacctcacgtgatacagcctatcgggatcctctcattcctggcgctggctcagagagacgggaagaggactcctttgacagtgatagcacagccaccttgctcaacacccggcccctgcaagacttgtctccatctagctcagcccaagccctggaggagctgtttccccgctacaccagccttcggccagggcctccactcaatcccccagattttcaggggctgagagatgcattggattcagagcatacccgccgcaagcattgtgagcgccatattcagagcctgcagaccctagtgttagagctacagcaacaattagccgtggctgtggctgccgaccgcaagaaagataccatgattgaacaactggacaagaccctggcccgtgtggtggagggctggaaccggcatgaggctgagcggacagaggttctcaggggacttcaagaggaacaccaggcagcagagctcaccagaagcaagcagcaggagacagtaacccgcctggaacaaagcctttctgaggccatggaggccctgaatcgtgagcaggaaagtgccagactgcagcaacgggaaagagagacactggaggaggaaaggcaagctctgactctgaggttggaggcagaacagcagcggtgctgtcgcctgcaggaagagcgggatgcagctcgggctgggcaactgagtgagcatcgagagttggagactcttcgggctgccctagaagaagaacggcagacctgggcccagcaagagcaccagcttaaggaacactaccaggcgctgcaggagaagagccaggctcagctggaaagagagaaggagaagagccagagagaagcccaggccgcctgggagacccagcaccagttggcattggtgcagtctgaggtgcggcggctggaaggagagctggatacagctcggagagagagagatgccctgcagctggaaatgagcttggtgcaggcccggtatgaaagccagcggatccagctggagtcggagctggctgtgcagctggagcagcgggtgacagagcggctggcgcaggctcaggagagcagcctacggcaagcagcctccctcagggaacatcacaggaagcagctgcaggacctgagtggacagcaccagcaggagctggccagtcagctagctcagttcaaggtggaaatggcagaacgagaggaacggcaacagcaggtggctgaggactacgagctcagactggcccgggagcaagcgcgagtgtgcgaactgcagagtgggaaccagcagctggaggagcagcgggtggagctggtggaaagactgcaggccatgctgcaggcccactgggatgaggccaaccagctgctcagcaccactctcccgccgcccaaccctccagctcctcctgctggaccctccagccccgggcctcaggagcccgagaaggaggagaggagagtctggactatgcctcccatggccgtggccctgaagcctgtattgcagcagagccgggaagcaagggacgagctacctggagcgcctcctgttctttgcagttcctcctcagatcttagcctcctgttgggcccctcttttcagagccagcattctttccagcccctggagcccaaaccagacctcacttcatccacagctggggccttctctgcacttggggccttccatcccgatcatagggcagaaaggccattccctgaggaagatcctggacctgacggggagggcctcctaaagcaagggctgccgcctgctcagctggagggcctcaagaattttttgcaccagttgctggagacagtgccccagaacaatgagaacccttctgtcgacctgttgccccctaagtctggtcctctgactgtcccatcttgggaggaagcccctcaagtgccacgtattccaccgcctgtccacaaaaccaaagttcccttagccatggcatccagtcttttccgggtccctgagcctccctcctcccattcacaaggcagtggtcccagcagtggttccccagagagaggtggagatgggcttacattcccaaggcagctgatggaggtgtctcaactgttgcgactctaccaggctcggggctggggggctctgcctgctgaggatctcctgctctacctgaagaggctggaacacagcgggtacaagcctgggaggaaggaggaaggattctccgggtggaagctggattatggggagtggagtgggactgatggccgaggggataatgtccccagaaggaacacagactcccgcttgggtgagatcccccggaaagagattccctcccaggctgtccctcgccgccttgctacagcccccaagactgaaaaacctcccgcacggaagaaaagtgggcaccctgccccgagtagcatgaggagccgggggggagtctggagatgagcccccctaccctctctcctctttgttctctcattgttgttattttaataaatgctcattagtctgcaaaaaaaaaaaaaaaaaaaa

SEQ ID NO: 5 - protein, 925 aa, Artificial Sequence
  Synthetic Peptide
MATSADSPSSPLGAEDLLSDSSEPPGLNQVSSEVTSQLYASLRLSRQAEATARAQLYLPSTSPPHEGLDGFAQELSRSLSVGLEKNLKKKDGSKHIFEMESVRGQLQTMLQTSRDTAYRDPLIPGAGSERREEDSFDSDSTATLLNTRPLQDLSPSSSAQALEELFPRYTSLRPGPPLNPPDFQGLRDALDSEHTRRKHCERHIQSLQTRVLELQQQLAVAVAADRKKDTMIEQLDKTLARVVEGWNRHEAERTEVLRGLQEEHQAAELTRSKQQETVTRLEQSLSEAMEALNREQESARLQQRERETLEEERQALTLRLEAEQQRCCVLQEERDAARAGQLSEHRELETLRAALEEERQTWAQQEHQLKEHYQALQEESQAQLEREKEKSQREAQAAWETQHQLALVQSEVRRLEGELDTARRERDALQLEMSLVQARYESQRIQLESELAVQLEQRVTERLAQAQESSLRQAASLREHHRKQLQDLSGQHQQELASQLAQFKVEMAEREERQQQVAEDYELRLAREQARVCELQSGNQQLEEQRVELVERLQAMLQAHWDEANQLLSTTLPPPNPPAPPAGPSSPGPQEPEKEERRVWTMPPMAVALKPVLQQSREARDELPGAPPVLCSSSSDLSLLLGPSFQSQHSFQPLEPKPDLTSSTAGAFSALGAFHPDHRAERPFPEEDPGPDGEGLLKQGLPPAQLEGLKNFLHQLLETVPQNNENPSVDLLPPKSGPLTVPSWEEAPQVPRIPPPVHKTKVPLAMASSLFRVPEPPSSHSQGSGPSSGSPERGGDGLTFPRQLMEVSQLLRLYQARGWGALPAEDLLLYLKRLEHSGYKPGRKEEGFSGWKLDYGEWSGTDGRGDNVPRRNTDSRLGEIPRKEIPSQAVPRRLATAPKTEKPPARKKSGHPAPSSMRSRGGVWR

SEQ ID NO: 6 - protein, 22 aa, Artificial Sequence
  Synthetic peptide
YKPGRKEEGFSGWKLDYGEWSG

SEQ ID NO: 7 - RNA, 19 nt, Artificial Sequence
  Synthetic Polynucleotide
agugccacacugcagcaac

SEQ ID NO: 8 - RNA, 19 nt, Artificial Sequence
  Synthetic Polynucleotide
cccucucuccucuuuguuc

SEQ ID NO: 9 - RNA, 19 nt, Artificial Sequence
  Synthetic Polynucleotide
cagucgcguuugcgacugg

SEQ ID NO: 10 - RNA, 19 nt, Artificial Sequence
  Synthetic Polynucleotide
cuggacuuccagaagaaca

(19) United States
(12) Patent Application Publication
(10) Pub. No.: US 2009/0137479 A1
(43) Pub. Date: May 28, 2009

(54) METHODS OF TREATING AND DIAGNOSING LAMINOPATHY

(76) Inventor: Qingshen Gao, Lake Forest, IL (US)

Correspondence Address: MARSHALL, GERSTEIN & BORUN LLP, 233 SOUTH WACKER DRIVE, 6300 SEARS TOWER, CHICAGO, IL, 60606-6357, US

(21) Appl. No.: 12/345,244

(22) Filed: Dec. 29, 2008

Related U.S. Application Data
(63) Continuation-in-part of application No. 11/992,813.
(60) Provisional application No. 60/722,752, filed on Sep. 30, 2005.

(51) Int. Cl. A61K 38/16; C12Q 1/02; G01N 33/68; C12Q 1/68; A61P 43/00

(52) U.S. Cl. 514/12; 435/29; 436/86; 435/6

(57) ABSTRACT

The invention provides a method of treating laminopathy in a subject comprising administering a therapeutically effective amount of centrobin polypeptide to the subject such that laminopathy is treated. The invention also provides a method of identifying an agent that enhances nuclear envelope integrity. The method comprises administering an agent to a cell comprising mutant centrobin, allowing the cell to replicate to form daughter cells, and observing nuclear envelope morphology within the daughter cells. In addition, the invention includes a method for diagnosing or identifying a predisposition to a laminopathy. The method comprises detecting the presence or absence of mutant centrobin in a sample, wherein the presence of mutant centrobin indicates that the subject is suffering from, or is predisposed to develop, a laminopathy. A kit for diagnosing or identifying a predisposition to a laminopathy also is provided.

### CROSS-REFERENCE TO RELATED APPLICATIONS AND INCORPORATION BY REFERENCE

This application is a continuation-in-part of U.S. patent application Ser. No. 11/992,813, filed Mar. 27, 2008, which is a national stage application filed under 35 U.S.C § 371 of International Patent Application No. PCT/US2006/038325, filed Oct. 2, 2006, which claims the benefit of U.S. Provisional Patent Application No. 60/722,752, filed Sep. 30, 2005. These patent applications are hereby incorporated by reference in their entireties.

### TECHNICAL FIELD OF THE INVENTION

The invention is in the field of protein therapeutics and methods of use thereof. More particularly, the field is protein therapeutics useful in treating diseases involving the abnormal nuclear envelopes characteristic of laminopathies.

### BACKGROUND OF THE INVENTION

Nuclear envelopathies encompass diseases caused by mutations in nuclear envelope proteins (Broers et al., Physiol. Rev., 86:967-1008 (2006)). Laminopathies are a subgroup of nuclear envelopathies involving defects in the nuclear lamina, a structure tightly associated with the inner nuclear membrane and essential for maintaining nucleus shape (Worman et al., Exp. Cell. Res., 313:2121-2133 (2007)). Nuclear lamina is mainly composed of lamin A/C and lamin B1/B2 and their binding proteins. Laminopathies are generally caused by mutations in the lamin A/C (LMNA) gene which encodes both lamin A and lamin C, or by mutations in genes involving prelamin A posttranslational processing (Broers et al., supra). Some laminopathies affect specific tissue types (e.g., striated muscle, peripheral nerves, or adipose tissue), while others act upon multiple types of tissues creating overlapping or systemic phenotypes.

Emery-Dreifuss muscular dystrophy (EDMD) is a laminopathy affecting striated muscle. The disease is characterized by (1) early joint contractures involving, e.g., elbows, Achilles tendons, and postcervical muscles; (2) progressive muscle weakness and wasting; and (3) cardiac involvement (Broers et al., supra; Emery et al., J. Med. Genet., 26:637-641 (1989); Rowland et al., Ann. Neurol, 5:111-117 (1979); Emery et al., J. Neurol. Neurosurg. Psychiatry, 29:338-342 (1966)). Dilated cardiomyopathy associated with EDMD presents as heart block with risk of sudden death. If patients suffering from symptoms of EDMD can be diagnosed early, pacemaker implantation can be life saving. There is currently no cure for laminopathies, including EDMD. Symptoms of the disease may be treated by, for example, physical therapy, corrective orthopedic surgery, pacemaker installation, and pharmaceutical intervention to, e.g., control seizures and the effects of lipodystrophy.

Identifying EDMD in a patient can be difficult using clinical parameters routinely used in muscular dystrophy diagnosis. Creatine kinase levels in EDMD patients are normal or moderately elevated (Broers et al., supra). Non-specific myopathic or dystrophic changes are typically seen in muscle biopsies, while diffuse patterns of muscle CT-scan involvement is observed in biceps, soleus, peroneal, external vasti, gluteus, and paravertebral muscles (Broers et al., supra). Genetic or molecular testing can suggest the specific laminopathy at issue. EDMD has been linked to causative mutations in two nuclear envelope protein genes, EMD (encoding emerin) and LMNA, in a minority of patients with EDMD or EDMD-related symptoms (Bione et al., Nat. Genet., 8:323-327 (1994); Bonne et al., Nat. Genet., 21:285-288 (1999)). However, the remaining 60% of the EDMD or EDMD-related cases are likely caused by mutations in other unidentified genes.

In view of the above, a need continues to exist in the art for methods of treating laminopathies and, in particular, EDMD. A need also exists for materials and methods for diagnosing laminopathies, such as EDMD. The invention provides such materials and methods.

### SUMMARY OF THE INVENTION

The invention provides a method of treating laminopathy in a subject. The method comprises administering a therapeutically effective amount of centrobin to the subject such that the laminopathy is treated. The centrobin is a centrobin polypeptide or biologically active fragment thereof, which may be expressed from a nucleic acid molecule administered to the subject. Laminopathies suitable for treatment include, for example, a laminopathic lipodystrophy disorder, a systemic laminopathy, a laminopathic neurological disorder, or a muscle laminopathy.

In one aspect, the invention provides a method of identifying an agent that enhances nuclear envelope integrity. The method comprises (a) administering an agent to a cell comprising mutant centrobin, (b) allowing the cell to replicate to form daughter cells, and (c) observing nuclear envelope morphology within the daughter cells. A reduction in nuclear envelope morphology defects in the daughter cells identifies an agent that enhances nuclear envelope integrity.

In addition, the invention includes a method for diagnosing or identifying a predisposition to a laminopathy. The method comprises detecting the presence or absence of mutant centrobin in a sample (e.g., a cellular sample) from a subject, wherein the presence of mutant centrobin indicates that the subject is suffering from, or is predisposed to develop, a laminopathy. A kit for diagnosing or identifying a predisposition to a laminopathy also is provided. The kit comprises a detection agent selected from the group consisting of nucleic acid primers suitable for amplifying a centrobin coding sequence that facilitates detection of a mutation; a nucleic acid probe specific for a mutant centrobin coding sequence, and an antibody or fragment thereof that selectively binds mutant centrobin. The kit further comprises instructions for detecting mutant centrobin.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. Where protein therapy is described, embodiments involving polynucleotide therapy (using polynucleotides/vectors that encode the protein) are specifically contemplated, and the reverse also is true.

In addition to the foregoing, the invention includes, as an additional aspect, all embodiments of the invention narrower in scope in any way than the variations specifically mentioned above. With respect to aspects of the invention described as a genus, all individual species are individually considered separate aspects of the invention. With respect to aspects of the invention described or claimed with “a” or “an,” it should be understood that these terms mean “one or more” unless context unambiguously requires a more restricted meaning. With respect to elements described as one or more within a set, it should be understood that all combinations within the set are contemplated. Additional features and variations of the invention will be apparent to those skilled in the art from the entirety of this application, and all such features are intended as aspects of the invention.

### DESCRIPTION OF THE FIGURES

FIG. 1 provides the nucleic acid sequence of centrobin a (SEQ ID NO: 1). The coding region spans nucleotides 920 through 3631. The coding region is provided as SEQ ID NO: 2.

FIG. 2 provides the deduced amino acid sequence of centrobin α (SEQ ID NO: 3).

FIG. 3 provides the nucleic acid sequence of centrobin β (SEQ ID NO: 4). A 66-base-pair in-frame insertion not found in centrobin α is shown in bold.

FIG. 4 provides the deduced amino acid sequence of centrobin β (SEQ ID NO: 5). A 22-amino-acid sequence not found in centrobin α (SEQ ID NO: 6) is shown in bold.

FIG. 5 is an illustration of the predicted coiled-coil regions of centrobin, ninein, and pericentrin.

FIG. 6 is a diagram of centrobin mutants correlated with their ability to bind to lamin A/C.

FIG. 7 is a diagram of lamin mutants correlated with their ability to bind to centrobin.

### DETAILED DESCRIPTION OF THE INVENTION

The invention is predicated, at least in part, on the surprising discovery that mutation of the gene encoding centrobin is associated with Emery-Dreifuss muscular dystrophy (EDMD). It was previously reported that centrobin is localized to the daughter centriole and is required for centriole duplication. However, as described herein, centrobin depletion unexpectedly results in grossly misshapen and herniated nuclei with disorganized nuclear envelope, a phenotype that is remarkably similar to that of fibroblast cells derived from EDMD patients with EMD or LMNA mutations. In addition, a deleterious centrobin mutation was observed in patients displaying EDMD phenotypes without harboring LMNA and EMD mutations.

In one aspect, the invention provides a method of treating laminopathy in a subject. The method comprises administering a therapeutically effective amount of centrobin polypeptide to the subject such that laminopathy is treated. Additionally, the invention provides a method of preventing laminopathy in a subject, wherein the method comprises administering a prophylactically effective amount of centrobin polypeptide to the subject such that the onset of laminopathy is prevented or delayed. By “therapeutic” or “treating” is meant the amelioration of the laminopathy, itself, and the protection, in whole or in part, against further progression of the laminopathy, in particular EDMD. By “prophylactic” or “preventing” or “inhibiting” is meant the protection, in whole or in part, against laminopathy, in particular EDMD, and symptoms associated therewith. “Preventing” also can entail slowing (or delaying) the onset of laminopathy in a subject. One of ordinary skill in the art will appreciate that any degree of protection from, or amelioration of, a laminopathy or symptom associated therewith is beneficial to a subject, such as a human patient. For example, the inventive method may reduce the severity of symptoms in a subject and/or delay the appearance of symptoms, which improves the quality of life of the subject.

Any laminopathy improved by administration of centrobin is suitable for prophylactic or therapeutic treatment by the inventive method. Laminopathies appropriate for treatment include, but are not limited to, laminopathic lipodystrophy disorders, systemic laminopathies, laminopathic neurological disorders, or muscle laminopathies. By “laminopathic” lipodystrophy disorders and “laminopathic” neurological disorders is meant lypodystrophy and neurological disorders resulting from or associated with abnormal nuclear envelope morphology. Lipodystrophy disorders are characterized by abnormal distribution of adipose tissue, optionally associated with metabolic disorders such as diabetes and hypertriglyceridemia. Lipodystrophy patients often experience selective loss and/or excessive accumulation of adipose tissue in certain regions of the body (e.g., loss in the limbs accompanied by excessive deposit in the upper back). Examples of laminopathic lipodystrophy disorders include, for instance, familial partial lipodystrophy (Dunnigan type), acquired partial lipodystrophy, type A insulin resistance syndrome, generalized lipoatrophy syndrome, and familial partial lipodystrophy (Kobberling).

Systemic laminopathies affect a variety of tissue types and include, e.g., atypical Werner syndrome, progeria (e.g., Hutchinson-Gilford progeria syndrome), restrictive dermopathy, and mandibuloacral dysplasia. The symptoms associated with systemic laminopathies are diverse. Atypical Werner syndrome patients prematurely exhibit features commonly associated with aging such as short stature, osteoporosis, thinning hair, athlerosclerosis, and cataracts. Restrictive dermopathy, on the other hand, is commonly associated with skin and joint contracture, abnormal skull mineralization, and pulmonary defects. Laminopathic neurological disorders, or laminopathies with peripheral nerve involvement, also are suitable for treatment by the inventive method. Neurological laminopathies include, e.g., Charcot-Marie-Tooth disease type 2B1, autosomal dominant leukodystrophy, and autosomal dominant spinal muscular dystrophy.

A majority of laminopathies caused by lamin A/C mutations involve striated muscle (Broers et al., supra). Emery-Dreifuss muscular dystrophy (EDMD), limb-girdle muscular dystrophy type 1B, congenital muscular dystrophy, multisystem dystrophy syndrome, dilated cardiomyopathy 1A, and dilated cardiomyopathy with conduction system defects are diagnosed as muscle laminopathies. Patients suffering from muscle laminopathies exhibit, for example, muscle weakness or wasting, hypertrophy of select muscles (e.g., calf), muscle or tendon contractures, cardiomyopathy, impaired cardiac conduction, and mental retardation. In one embodiment, the inventive method comprises treating or preventing Emery-Dreifuss muscular dystrophy type 2.

### Centrobin

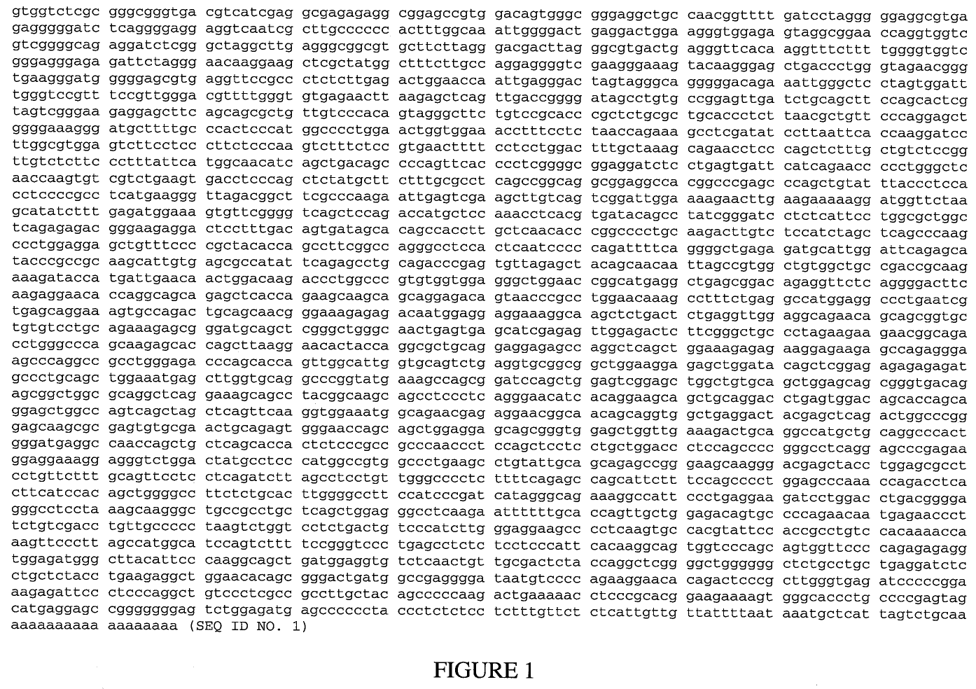

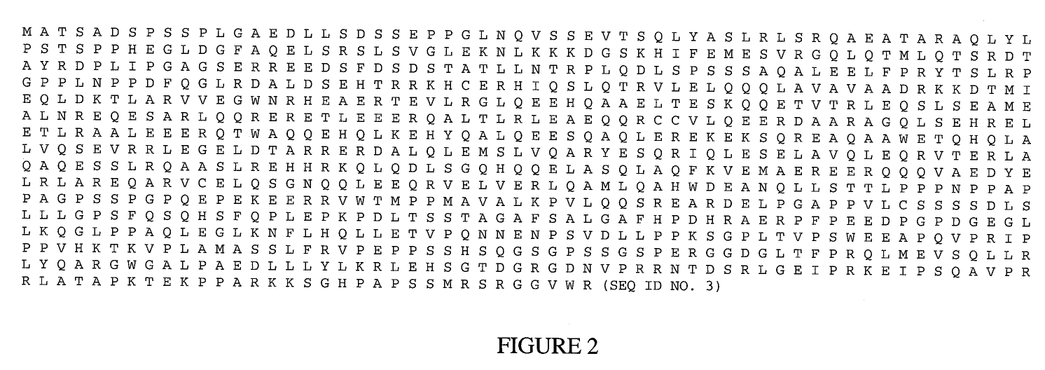

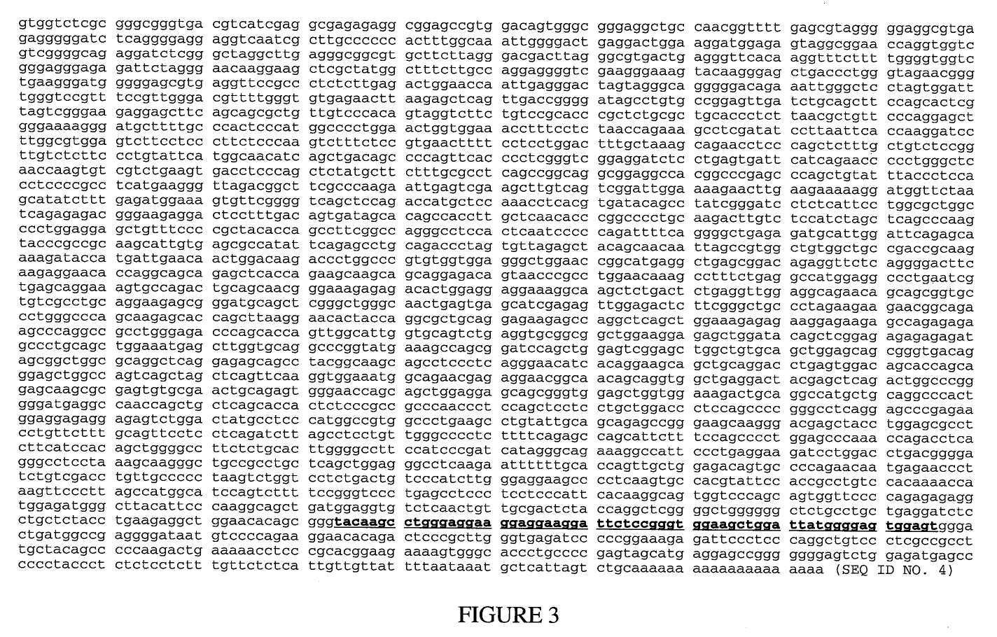

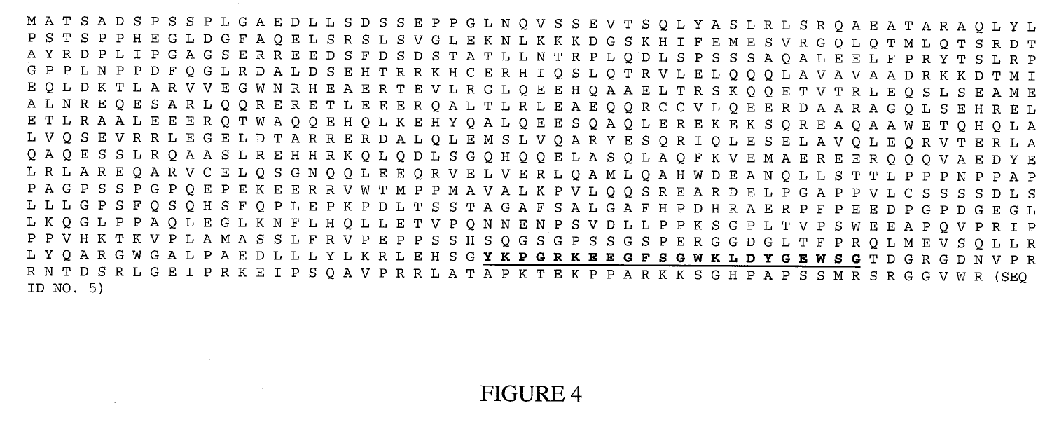

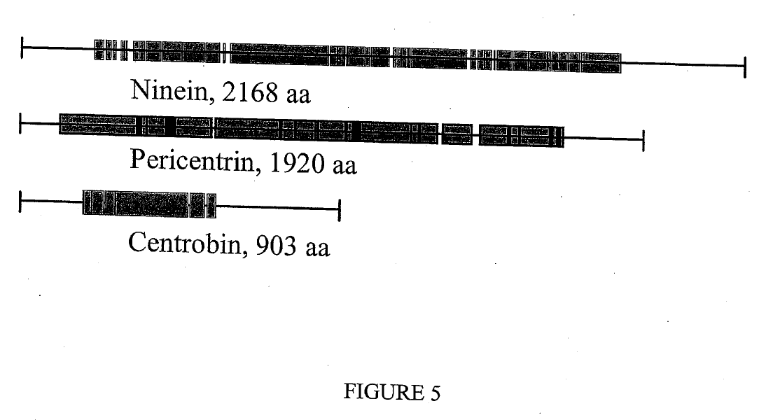

Molecular cloning of full-length centrobin identified two transcripts. The major transcript is encoded by the nucleic acid sequence of SEQ ID NO: 1 (Accession No. AY160226; illustrated in FIG. 1). The coding region of SEQ ID NO: 1 is provided in SEQ ID NO: 2, which encodes a polypeptide comprising the amino acid sequence of SEQ ID NO: 3 (illustrated in FIG. 2). The minor transcript is encoded by the nucleic acid sequence of SEQ ID NO: 4 (Accession No. AY160227; illustrated in FIG. 3), which encodes a polypeptide comprising the amino acid sequence of SEQ ID NO: 5 (illustrated in FIG. 4). Sequence comparison of the two transcripts revealed that the minor transcript contains a 66 base pair in-frame insertion after nucleotide 3433 of the major transcript, shown in bold in FIG. 3, which encodes a 22 amino acid sequence (SEQ ID NO: 6) shown in bold in FIG. 4. The major transcript, which comprises 3718 nucleotides, is referred to as “centrobin α” while the minor transcript, which comprises 3784 nucleotides, is referred to as “centrobin β.” Sequence analysis showed that centrobin shares homology to various coiled-coiled proteins, including two centrosomal proteins, pericentrin and ninein. An alpha helical domain is located at amino acids 189-619 of centrobin α and centrobin β. Similar to pericentrin and ninein, centrobin is predicted to have a coiled-coil region in the middle and non-coiled regions at both the C-terminal end and the N-terminal end (FIG. 5). Centrobin is further described in U.S. patent application Ser. No. 11/992,813 and International Patent Publication WO 2007/041439, both of which are incorporated by reference in their entirety and specifically with respect to centrobin peptide and nucleic acid descriptions. Centrobin also is further described in Zou, et al., J. Cell Biol., 171 (3):437-445 (2005), which is hereby incorporated by reference.

Certain embodiments of the invention entail administering centrobin to a subject to treat or prevent a laminopathy. Centrobin may be administered in any suitable form, e.g., a centrobin peptide is administered to the subject and/or a polynucleotide encoding centrobin is administered to induce production of centrobin polypeptide within the body. The centrobin peptide preferably comprises the amino acid sequence set forth in SEQ ID NO: 3 or SEQ ID NO: 5. Alternatively, a centrobin peptide may comprise an amino acid sequence that differs in amino acid sequence from that of SEQ ID NO: 3 or SEQ ID NO: 5 due to natural allelic variation or mutagenesis, but retains the functional activity of a peptide comprising the amino acid sequence of SEQ ID NO: 3 or SEQ ID NO: 5. In this regard, a practitioner can modify a centrobin polypeptide to create a functional variant falling within the scope of the invention using routine laboratory techniques. Exemplary amino acid substitutions are those which reduce susceptibility to proteolysis, reduce susceptibility to oxidation, and/or confer or modify other physiochemical or functional properties of centrobin polypeptides. According to certain embodiments, single or multiple amino acid substitutions (in certain embodiments, conservative amino acid substitutions) are made in the naturally-occurring sequence.

For example, the centrobin peptide can comprise one or more conservative amino acid substitutions within the amino acid sequence of SEQ ID NO: 3 or SEQ ID NO: 5. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, and histidine), acidic side chains (e.g., aspartic acid and glutamic acid), uncharged polar side chains (e.g., glycine, asparagine, glutamine, serine, threonine, tyrosine, and cysteine), nonpolar side chains (e.g., alanine, valine, leucine, isoleucine, proline, phenylalanine, methionine, and tryptophan), beta-branched side chains (e.g., threonine, valine, and isoleucine) and aromatic side chains (e.g., tyrosine, phenylalanine, tryptophan, and histidine). It will be appreciated, however, that a practitioner is not limited to creating conservative substitutions so long as the resulting polypeptide retains one or more functions of a polypeptide encoded by SEQ ID NOS: 3 or 5, such as the ability to reduce severity of nuclear envelope defects associated with laminopathies. To this end, the amino acid substitution preferably does not substantially change the structural characteristics of the parent sequence (e.g., disrupt secondary structure characterizing the parent sequence). Examples of art-recognized polypeptide secondary and tertiary structures are described in, e.g., Proteins, Structures and Molecular Principles, Creighton, Ed., W. H. Freeman and Company, New York (1984); Introduction to Protein Structure, C. Branden and J. Tooze, Eds., Garland Publishing, New York, N.Y. (1991); and Thornton et al., Nature, 354:105 (1991), which are each incorporated herein by reference. In addition, the invention includes use of a naturally occurring centrobin allelic variant or isoform resulting from alternate RNA splicing.

The inventive method may comprise administering a polypeptide (or a polynucleotide encoding a polypeptide) having an amino acid sequence that is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NOS: 3 or 5 and that reduces nuclear envelope defects in laminopathy-afflicted cells. “Sequence identity” means that two amino acid or polynucleotide sequences are identical over a region of comparison, such as a region of at least about 250 residues of SEQ ID NOS: 3 or 5. Optionally, the region of identity spans at least about 500-750 residues or over at least about 900 residues of SEQ ID NO: 3 and 5 (e.g., the full-length sequence), and spans the active domain of the polypeptide. Several methods of conducting sequence alignment are known in the art and include, for example, the homology alignment algorithm (Needleman & Wunsch, J. Mol. Biol., 48:443 (1970)); the local homology algorithm (Smith & Waterman, Adv. Appl. Math., 2:482 (1981)); and the search for similarity method (Pearson & Lipman, Proc. Natl. Acad. Sci. USA, 85:2444 (1988)). An algorithm widely used to determine percent sequence identity and sequence similarity is the BLAST algorithm (Altschul et al., J. Mol. Biol., 215:403-410 (1990); Henikoff & Henikoff. Proc. Natl. Acad. Sci. USA, 89:10915 (1989); Karlin & Altschul, Proc. Natl. Acad. Sci. USA, 90:5873-5787 (1993)). Software for performing BLAST analyses is publicly available through the National Center for Biotechnology Information. Other examples of alignment software include GAP, BESTFIT, FASTA, PILEUP, and TFASTA provided by Wisconsin Genetics Software Package (Genetics Computer Group, 575 Science Dr., Madison, Wis.), and CLUSTALW (Thompson et al., Nuc. Acids Res., 22:4673-4680 (1994); http://www.ebi.ac.uk/Tools/clustalw2/index.html).

If desired, the centrobin polypeptide administered to the subject is a biologically-active portion (i.e., fragment) of a full-length centrobin polypeptide (i.e., a polypeptide comprising the amino acid sequence of SEQ ID NO: 3 or 5). A biologically-active centrobin fragment can comprise, e.g., 250, 500, 600, 700, 800, 900 or more amino acids of the coding sequence set forth in SEQ ID NO: 3 or 5, so long as the fragment retains the ability to reduce nuclear envelope defects. In this regard, a biologically active centrobin fragment binds lamin A/C and comprises a nuclear localization signal. Full-length, wild-type centrobin comprises an active nuclear localization signal at its C-terminus. In the context of the invention, a centrobin polypeptide may be truncated at the N- and/or C-terminus to create a biologically-active centrobin fragment, such as a fragment comprising amino acids 371-903 of SEQ ID NO: 3. A biologically-active centrobin fragment (or a full length centrobin polypeptide) may be operably linked to a non-centrobin peptide to create a fusion protein. The non-centrobin portion of the fusion protein can be linked to the N- or C-terminus of the centrobin polypeptide, inserted within the centrobin polypeptide, or can replace a portion of the centrobin polypeptide, so long as the fusion protein retains biological activity. A centrobin chimeric or fusion protein can be produced by standard recombinant DNA techniques and using automated DNA synthesizers. Many expression vectors encoding a fusion moiety (e.g., a glutathione S-transferase (GST) polypeptide or His tag) are commercially available.

Any method of obtaining or constructing peptides is proper in the context of the invention so long as the centrobin polypeptide is active and suitable for administration to a subject. For example, wild-type centrobin peptides can be isolated from cell or tissue sources using standard protein purification techniques. Alternatively, centrobin peptides are produced by recombinant DNA techniques or synthesized chemically. By “isolated” is meant that the peptide (or nucleic acid) is separated from other peptides (or nucleic acids) that are present in the natural source of the peptide, or substantially free from chemical precursors or other chemicals when chemically synthesized. In one aspect, “substantially free” of, e.g., cellular material, includes preparations of peptide having less than about 30% (by dry weight) of non-centrobin peptide, as further described in U.S. patent application Ser. No. 11/992,813, and International Patent Publication WO 2007/041439 (both of which are incorporated by reference). Homologous centrobin polypeptides of species other than humans, including, but not limited to mouse, rat, rabbit, dog, cat, cow, horse, primate, having activity similar to that of human centrobin and having acceptable immunogenicity also are contemplated.

### Centrobin Nucleic Acids

Nucleic acid molecules that encode centrobin polypeptides or biologically active portions thereof are suitable for use in the context of the invention. For example, the method of treating a laminopathy can entail administering to the subject a nucleic acid that expresses a centrobin polypeptide to promote centrobin production within the subject. As used herein, the terms “nucleic acid molecule” and “nucleic acid” are intended to include DNA molecules (e.g., cDNA or genomic DNA), RNA molecules (e.g., mRNA), analogs of DNA or RNA generated using nucleotide analogs (e.g., nucleic acid sequences having a structure similar to the native compound but comprising different components or side chains), and the like. When treating or preventing a laminopathy, the nucleic aid molecule (i.e., polynucleotide) is preferably a DNA molecule. In some aspects of the invention, the nucleic acid molecule comprises a nucleic acid sequence encoding the amino acid sequence set forth in SEQ ID NO: 3 or SEQ ID NO: 5 (or biologically-active fragments thereof). The nucleic aid molecule may comprise the nucleic acid sequence set forth in SEQ ID NO: 1, SEQ ID NO: 2, or SEQ ID NO: 4. Alternatively, the nucleic acid molecule comprises one or more modified nucleotides or nucleotide substitutions compared to SEQ ID NO: 1, SEQ ID NO: 2, or SEQ ID NO: 4 thereby encoding a centrobin polypeptide variant as discussed above. Nucleic acids suitable for use in the context of the invention include, but are not limited to, those comprising a nucleic acid sequence containing regions that are at least about 30%, 50%, 60%, 70%, 75%, 80%, 85%, 90%, 95%, 97%, 98% or 99% identical to a region of SEQ ID NO: 1, SEQ ID NO: 2, or SEQ ID NO: 4 of identical size. Preferably, the polynucleotide comprises a nucleic acid sequence exhibiting the above-recited percent identity over the entire coding region of SEQ ID NO: 1, SEQ ID NO: 2, or SEQ ID NO: 4.

In some embodiments, it may be advantageous to incorporate the nucleic acid into an expression vector. One of ordinary skill in the art will appreciate that any of a number of expression vectors known in the art are suitable for use in the context of the invention, such as, but not limited to, plasmids, plasmid-liposome complexes, and viral vectors. Any of these expression vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Expression vectors, nucleic acid regulatory sequences, administration methods, and the like, are further discussed in U.S. Patent Publication No. 20030045498. Expression vectors encoding centrobin are further described in U.S. patent application Ser. No. 11/992,813, and International Patent Publication WO 2007/041439.

Certain embodiments of the invention may employ nucleic acid fragments (oligonucleotides) that bind a centrobin coding sequence or the complement thereof. Oligonucleotides are useful as primers for the amplification of centrobin nucleic acid molecules. In this regard, oligonucleotides for use in the invention ideally comprise a sufficient number of nucleotide bases to be used in a polymerase chain reaction (PCR) reaction, and can be based on, or designed from, a genomic or cDNA sequence. Oligonucleotides also are useful as hybridization probes to identify (i.e., confirm or reveal) the presence of nucleic acids encoding wild-type or mutant centrobin in a sample. “Probes” refer to nucleic acids derived from any contiguous portion of a nucleic acid sequence of choice. The length of a probe is preferably sufficient for specific hybridization to a nucleic acid sequence encoding centrobin (e.g., wild-type centrobin or mutant centrobin). A nucleic acid probe can comprise as few as 5, 6, 7, 8, 9, or 10 nucleotides that bind to a nucleic acid sequence encoding centrobin (or the complement thereof). Preferably, probes are about 15-100 nucleotides in length (e.g., about 25, 30, 50, or 75 nucleotides), although probes can comprise as many as about 250, 500, 1000, or 3,000 nucleotides, depending on the desired specificity and conditions of the hybridization reaction.

Short nucleic acid molecules are easily generated synthetically, while longer polynucleotides may be obtained from a natural or recombinant source. Probes may be single- or double-stranded, and preferably are designed to have specificity in PCR, membrane-based hybridization technologies, or ELISA-like technologies. In some embodiments, a probe comprises a label attached thereto, e.g., the probe is labeled with a radioisotope, a fluorescent compound, a biotin-avidin label, an enzyme, or an enzyme co-factor. A non-limiting example of a probe for detecting mRNA or genomic DNA is a labeled nucleic acid probe of sufficient length to specifically hybridize under stringent conditions to mRNA or genomic DNA. Centrobin peptides and nucleic acids comprising a nucleic acid sequence encoding a centrobin peptide or biologically-active fragment thereof are further described in International Patent Publication WO 2007/041439 (which is hereby incorporated by reference).

### Administration Considerations

The inventive method is preferably performed as soon as possible after it has been determined that a subject is at risk for developing a laminopathy (e.g., diagnosis of close family member) or as soon as possible after onset of the laminopathy is detected. To this end, centrobin is administered before symptoms appear to protect, in whole or in part, against the onset of laminopathy. Centrobin also can be administered after symptoms are detected to prevent, in whole or in part, additional symptoms or an increase in symptom severity.

A particular administration regimen for a subject will depend, in part, upon the form of centrobin administered (e.g., polypeptide or nucleic acid molecule), the amount administered, the route of administration, and the cause and extent of any side effects. The amount of centrobin administered to a subject (e.g., a mammal, such as a human) in accordance with the invention should be sufficient to effect the desired response over a reasonable time frame. Dosage typically depends upon a variety of factors, including the particular agent employed, the age and body weight of the subject, as well as the existence of any disease or disorder in the subject. The clinician may titer the dosage and modify the route of administration to obtain the optimal therapeutic effect, and conventional range-finding techniques are known to those of ordinary skill in the art. Purely by way of illustration, the inventive method can comprise administering, e.g., from about 0.1 μg/kg to up to about 100 mg/kg of centrobin or more, depending on the factors mentioned above. In other embodiments, the dosage may range from 1 μg/kg up to about 100 mg/kg; or 5 μg/kg up to about 100 mg/kg; or 10 μg/kg up to about 100 mg/kg. Some conditions or disease states require prolonged treatment, which may or may not entail administering lower doses of agent over multiple administrations. In addition, when appropriate, centrobin is administered in combination with other substances (e.g., therapeutics) and/or other therapeutic modalities to achieve an additional (or augmented) biological effect.

Suitable methods of administering a physiologically acceptable composition, such as a pharmaceutical composition comprising a centrobin polypeptide (or DNA encoding a centrobin polypeptide), are well known in the art. Although more than one route can be used to administer an agent, a particular route can provide a more immediate and more effective reaction than another route. Depending on the circumstances, a pharmaceutical composition comprising centrobin is applied or instilled into body cavities, absorbed through the skin or mucous membranes, ingested, inhaled, and/or introduced into circulation. For example, in certain circumstances, it will be desirable to deliver a pharmaceutical composition comprising centrobin orally, through injection by intravenous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, the agent is administered regionally via intraarterial or intravenous administration feeding a region of interest. Alternatively, the composition is administered locally via implantation of a membrane, sponge, or another appropriate material on to which the desired molecule has been absorbed or encapsulated. Where an implantation device is used, the device may be implanted into any suitable tissue or organ, and delivery of centrobin polypeptide or polynucleotide may be via diffusion, timed-release bolus, or continuous administration. In certain aspects of the invention, the centrobin polypeptide (or the polynucleotide encoding centrobin) is administered to the subject via direct injection into bone marrow or direct injection into muscle. Therapeutic delivery approaches are well known to the skilled artisan, some of which are further described, for example, in U.S. Pat. No. 5,399,363.

To facilitate administration, a protein or nucleic acid molecule can be formulated into a physiologically-acceptable composition comprising a carrier (i.e., vehicle, adjuvant, or diluent). The particular carrier employed is limited only by chemico-physical considerations, such as solubility and lack of reactivity with the therapeutic, and by the route of administration. Physiologically-acceptable carriers are well known in the art. Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, Banker and Chalmers. eds., J. B. Lippincott Co., Philadelphia. Pa., pages 238-250 (1982), and ASHP Handbook on Injectable Drugs, Toissel, 4th ed., pages 622-630 (1986)). A pharmaceutical composition comprising centrobin protein or centrobin nucleic acid molecule may be placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

The inventive method's efficacy in treating or preventing a laminopathy may be determined by monitoring one or more of the symptoms associated with a particular laminopathy. For example, when the subject is afflicted with Emery-Dreifuss muscular dystrophy type 2, the inventive method preferably improves muscle strength, reduces muscle wasting, or reduces cardiomyopathy in the subject. Muscle strength and wasting is determined using any of a number of clinically-acceptable techniques and protocols, such as computed tomography, electromyography, muscle biopsy, ultrasonography, and manual muscle strength testing (e.g., dynamometer testing). See, e.g., Musculoskeletal Assessment, H. Clarkson, Lippincott Williams & Wilkins (1999). Likewise, the existence and severity of cardiomyopathy can be examined via blood tests, x-ray, electrocardiogram, echocardiography, exercise testing, cardiac catheterization, magnetic resonance imaging, and measuring subject's left ventricular ejection fraction. It will be appreciated that the laminopathy symptoms described herein are merely exemplary. The clinical parameters used to diagnose and track the progression of laminopathies are well known in the art and described in, e.g., Broers et al., Physiol. Rev., 86:967-1008 (2006). In addition to physiological indications, quality of life parameters may signal the effectiveness of inventive method. In this regard, the patient may experience less muscle pain, exhibit increased physical endurance, and/or show improved physical appearance.

### Diagnostic Methods

The invention also includes a method for diagnosing or identifying a predisposition to a laminopathy. The method comprises detecting the presence or absence of mutant centrobin in a sample from a subject, wherein the presence of mutant centrobin indicates that the subject is suffering from, or is predisposed to develop, a laminopathy. The laminopathy may be a laminopathic lipodystrophy disorder, a systemic laminopathy, or a laminopathic neurological disorder. In a specific aspect of the invention, the laminopathy is a muscle laminopathy (e.g., Emery-Dreifuss muscular dystrophy (such as Emery-Dreifuss muscular dystrophy type 2), limb-girdle muscular dystrophy type 1B, congenital muscular dystrophy, multisystem dystrophy syndrome, dilated cardiomyopathy 1A, or dilated cardiomyopathy with conduction system defects). While detection of mutant centrobin may not, by itself, absolutely predict development of a particular disease, the presence or absence of centrobin mutants indicates an increased and/or decreased likelihood that a subject will develop symptoms associated with a laminopathy. This information is extremely valuable, and allows a subject to perform regular physical exams to monitor the progress and/or appearance of symptoms at an early stage.

The diagnostic method entails detecting mutant centrobin in a biological sample from a subject. Numerous methods of obtaining subject samples are widely used in the art and are appropriate in the context of the invention. Samples typically are isolated from blood, serum, urine, amniotic fluid, or tissue biopsies from, e.g., muscle, connective tissue, nerve tissue, placenta, and the like. If the subject is a fetus, a sample can be obtained by amniocentesis or chorionic villus sampling. Once obtained, cells from the sample are examined to detect the presence or absence of mutant centrobin. “Mutant centrobin” encompasses a centrobin polypeptide (or DNA or mRNA encoding a centrobin polypeptide) correlated or associated with a laminopathy phenotype, such as misshapen and herniated nuclei and/or regions of nuclear envelope pile-up. Mutant centrobin may fail to localize to the nucleus or exhibit impaired binding to lamin A/C, both of which result in a reduced ability to mediate organized nuclear envelopes compared to wild-type centrobin. In a specific embodiment, the method comprises detecting an N-terminal fragment of wild-type centrobin (e.g., a fragment caused by a thymidine deletion at base pair 75 of the nucleic acid sequence encoding centrobin α (SEQ ID NO: 2)), a non-wild-type amino acid corresponding to position 180 of SEQ ID NO: 3 (i.e., the amino acid residue that corresponds to position 180 of SEQ ID NO: 3 is not a proline), a non-wild-type amino acid corresponding to position 480 of SEQ ID NO: 3 (i.e., the amino acid residue that corresponds to position 480 of SEQ ID NO: 3 is not a histidine), a non-wild-type amino acid corresponding to position 539 of SEQ ID NO: 3 (i.e., the amino acid residue that corresponds to position 539 of SEQ ID NO: 3 is not an asparagine), a non-wild-type amino acid corresponding to position 870 of SEQ ID NO: 3 (i.e., the amino acid residue that corresponds to position 870 of SEQ ID NO: 3 is not an arginine), a frameshift in a location corresponding to exon 19 of wild-type centrobin, and/or incorrect splicing during expression relative to wild-type splicing of centrobin, all of which have been surprisingly identified in laminopathy patients. In certain embodiments wherein a nucleic acid sequence encoding mutant centrobin is examined, the method comprises detecting a deletion of thymidine at nucleotide 75 of SEQ ID NO: 2 (the coding sequence for centrobin α) (“75delT”), substitution of cytosine with adenine at nucleotide 539 of SEQ ID NO: 2 (“539C>A”), substitution of cytosine with thymidine at nucleotide 689 of SEQ ID NO: 2 (“689C>T”), substitution of adenine with thymidine at nucleotide 1439 of SEQ ID NO: 2 (“1439A>T”), substitution of adenine with thymidine at nucleotide 1615 of SEQ ID NO: 2 (“1615A>T”), substitution of guanine with adenine at nucleotide −12 of intron 16 (i.e., the substituted nucleotide is 12 nucleotides before position 1381 of the coding region of centrobin α, which is the starting nucleotide of exon 17) (“1381-12G>A”), substitution of guanine with adenine at nucleotide 2512 of SEQ ID NO: 2 (“2515G>A”), deletion of guanine at nucleotide 2699 of SEQ ID NO: 2 (“2699delG”), or substitution of cytosine with thymidine at nucleotide 2608 of SEQ ID NO: 2 (“2608C>T”).

It will be appreciated that mutant centrobin can be detected in a variety of ways. In one embodiment, the method comprises obtaining nucleic acid sequence data from the cellular sample. Suitable methods of directly analyzing a nucleic acid molecule include, for instance, denaturing high pressure liquid chromatography (DHPLC), DNA hybridization, computational analysis, automated fluorescent sequencing, clamped denaturing gel electrophoresis (CDGE), denaturing gradient gel electrophoresis (DGGE), mobility shift analysis, restriction enzyme analysis, heteroduplex analysis, chemical mismatch cleavage (CMC), RNase protection assays, use of polypeptides that recognize nucleotide mismatches, and direct manual sequencing. These and other methods are described in the art (see, for instance, Tabone et al., Nature Protocols, 1:2297-2304 (2006); MacBeath et al., DNA Sequencing Protocols, 167:119-152 (2001) (DOI: 10.1385/1-59259-113-2:119); Li et al., Nucleic Acids Research, 28(2):el (i-v) (2000); Liu et al., Biochem. Cell Bio., 80:17-22 (2000); Burczak et al., Polymorphism Detection and Analysis, Eaton Publishing (2000); Sheffield et al., Proc. Natl. Acad. Sci. USA, 86:232-236 (1989); Orita et al., Proc. Natl. Acad. Sci. USA, 86:2766-2770 (1989); Church and Gilbert, Proc. Natl. Acad. Sci. USA, 81:1991-1995 (1988); Cotton et al., Proc. Natl. Acad. Sci. USA, 85:4397-4401 (1985); Myers et al., Science, 230:1242-1246 (1985); Geever et al., Proc. Natl. Acad. Sci. USA, 78:5081-5085 (1981); Flavell et al., Cell, 15:25-41 (1978); Sanger et al., Proc. Natl. Acad. Sci. USA, 74:5463-5467 (1977); and U.S. Pat. No. 5,288,644).

In one embodiment, diagnosis of (or identification of a predisposition to) laminopathy can be accomplished using a hybridization method (see Current Protocols in Molecular Biology, Ausubel et al., eds., John Wiley & Sons (2007), including all supplements). A biological sample of genomic DNA, RNA, or cDNA is obtained from a subject suspected of having, being susceptible to, or experiencing symptoms associated with laminopathy. Optionally, the nucleic acid encoding centrobin is amplified by polymerase chain reaction (PCR). The DNA, RNA, or cDNA sample is then examined. The presence of mutant centrobin can be determined by sequence-specific hybridization of a nucleic acid probe specific for particular mutation within the centrobin coding sequence. As discussed above, a nucleic acid probe is a DNA molecule or an RNA molecule that hybridizes to a complementary sequence in genomic DNA, RNA, or cDNA. In some aspects, the presence of more than one centrobin mutation is determined by using multiple nucleic acid probes, each being specific for a particular mutation. In this regard, the inventive method can comprise detecting an N-terminal fragment of wild-type centrobin, detecting a codon encoding a non-wild-type amino acid corresponding to a position in SEQ ID NO: 3 selected from the group consisting of position 180, position 480, position 539, and position 870, and/or detecting a frameshift in exon 19 of wild-type centrobin.

One of skill in the art has the requisite knowledge and skill to design a probe so that sequence-specific hybridization will occur only if a particular mutation is present in a centrobin coding sequence. By “sequence-specific hybridization” is meant that the probe(s) preferentially bind to a nucleic acid sequence encoding mutant centrobin. In some embodiments, specific hybridization is achieved using “stringent conditions,” which are conditions for hybridization and washing under which nucleotide sequences at least 60% identical to each other typically remain hybridized. It is appreciated in the art that stringent conditions can differ depending on sequence content, probe length, and the like. Generally, stringent conditions are selected to be about 5° C. lower than the thermal melting point (Tm) for a specific sequence at a defined ionic strength and pH. Tm is the temperature (under defined ionic strength, pH, and nucleic acid concentration) at which 50% of the probes complementary to the target sequence hybridize to the target sequence at equilibrium. Since target sequences are generally present at excess, 50% of the probes are occupied at equilibrium at Tm. Stringent conditions also may include a salt concentration less than about 1.0 M sodium ion, typically about 0.01 to 1.0 M sodium ion (or other salts) at pH 7.0 to 8.3 and the temperature is at least about 30° C. for short probes, primers, or oligonucleotides (e.g., 10 nucleotides to 50 nucleotides) and at least about 60° C. for longer probes, primers and oligonucleotides. Stringent conditions may also be achieved with the addition of destabilizing agents, such as formamide. A non-limiting example of stringent hybridization conditions are hybridization in a high salt buffer comprising 6×SSC, 50 mM Tr-is-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C.

Specific hybridization, if present, is detected using standard methods. For example, the probe can comprise a fluorescent moiety at its 3′ terminus, a quencher at its 5′ terminus, and an enhancer oligonucleotide to facilitate detection, as described by Kutyavin et al., Nucleic Acid Res. 34:e128 (2006). In this detection method, an enzyme cleaves the fluorescent moeity from a fully complementary detection probe, but does not cleave the fluorescent moeity if the probe contains a mismatch. The presence of a particular target sequence is signalled by the fluorescence of the released fluorescent moiety. Alternatively, nucleic acids encoding centrobin are dot-blotted using standard methods, and the blot is contacted with one or more oligonucleotide probes specific for a centrobin mutation (see, for example, Saiki et al., Nature, 324:163-166 (1986)). Similarly, arrays of oligonucleotide probes complementary to target nucleic acid sequence(s) can be employed in the inventive diagnostic method. Oligonucleotide arrays typically comprise a plurality of different oligonucleotide probes coupled to a surface of a substrate (e.g., plastic, complex carbohydrate, or acrylic resin) in different known locations. Such arrays are generally produced using mechanical synthesis methods or light-directed synthesis methods, although other other methods are known to the ordinary skilled practitioner (see, e.g., Bier et al., Adv. Biochem. Eng. Biotechnol., 109:433-53 (2008); Hoheisel, Nat. Rev. Genet., 7:200-10 (2006); Fan et al., Methods Enzymol., 410:57-73 (2006); Raqoussis & Elvidge, Expert Rev. Mol. Diagn., 6:145-52 (2006); Mockler et al., Genomics, 85:1-15 (2005), and references cited therein, the entire teachings of each of which are incorporated by reference herein). In another hybridization method, Northern analysis (see Current Protocols in Molecular Biology, Ausubel et al., eds., John Wiley & Sons (2007)) is used to identify the presence of mutant centrobin encoded by RNA in a subject's sample. Specific hybridization between the nucleic acid probe and the nucleic acid in the subject sample indicates that mutant centrobin is present, and the subject is suffering from or is at risk of developing a laminopathy.

Sequence analysis can also be used to detect specific centrobin mutations associated with laminopathy. Therefore, in one embodiment, determination of the presence or absence of mutant centrobin entails directly sequencing DNA or RNA obtained from a subject. If desired, PCR is used to amplify a portion of a nucleic acid encoding centrobin, and the presence of a specific mutation is detected directly by sequencing the relevant site(s) of the DNA or RNA in the sample, e.g., the region encoding the N-terminal fragment of wild-type centrobin; the region encoding an amino acid residue corresponding to position 180, position 480, position 539, and/or position 870 of SEQ ID NO: 1 or SEQ ID NO: 2; or the region corresponding to exon 19 of wild-type centrobin.

Mutations in the centrobin coding sequence may lead to altered expression levels, e.g., a decrease in the expression level of an mRNA or protein, which lead to an abnormal phenotype. Such mutations are detected via, e.g., ELISA, radioimmunoassays, immunofluorescence, Northern blotting, and Western blotting to compare centrobin expression levels in a subject compared to a biologically-matched control or reference. These processes are described in, for instance, Sambrook et al., Molecular Cloning: A Laboratory Manual, 3^{rd} ed., Cold Spring Harbor Laboratory Press, Cold Spring Harbor, N.Y. (2001).

Alternatively or in addition, the diagnostic method entails detecting variant centrobin protein comprising an altered amino acid sequence (e.g., one or more deletions, substitutions, additions, and/or truncation) compared to wild-type centrobin. Any method of detecting mutant proteins is appropriate for use in the context of the invention, and many are known in the art. For example, centrobin may be isolated from a cellular sample and subjected to amino acid sequencing, the results of which are compared to a reference amino acid sequence. Mutant centrobin also can be identified by detecting altered molecular weight compared to wild-type centrobin using gel electrophoresis (e.g., SDS-PAGE). Immunoassays, e.g., immunofluorescent immunoassays, immunoprecipitations, radioimmunoasays, ELISA, and Western blotting, also can be used.

Several detection methods are accomplished using an anti-centrobin antibody or fragment thereof that selectively (or preferentially) binds mutant centrobin. The term “antibody” refers to a complete (intact) antibody (immunoglobulin) molecule (including polyclonal, monoclonal, chimeric, humanized, or human versions having full length heavy and/or light chains) or a centrobin binding fragment thereof. Antibody fragments include F(ab′)2, Fab, Fab′, Fv, Fc, and Fd fragments, and can be incorporated into single domain antibodies, single-chain antibodies, maxibodies, minibodies, intrabodies, diabodies, triabodies, tetrabodies, v-NAR and bis-scFv (see, e.g., Hollinger and Hudson, Nature Biotechnology, 23(9): 1126-1136 (2005)). Antibody polypeptides, including monobodies, also are disclosed in U.S. Pat. No. 6,703,199. Other antibody polypeptides are disclosed in U.S. Patent Publication No. 20050238646. Anti-centrobin antibodies are further described in U.S. patent application Ser. No. 11/992,813, and International Patent Publication WO 2007/041439, both of which are incorporated by reference.

The term “selectively binds” refers to the ability of the antibody or fragment thereof to bind to mutant centrobin with greater affinity (e.g., at least 10, 15, 20, 25, 50, 100, 250, 500, 1000, or 10,000 times greater affinity) than it binds to an unrelated control protein, such as hen egg white lysozyme. Preferably, the antibody distinguishes mutant centrobin from wild-type centrobin. Binding affinity can be determined using any of a number of methods known in the art such as an affinity ELISA assay, a BIAcore assay (i.e., a surface plasmon resonance-based assay), a kinetic method, or an equilibrium/solution method.

Various procedures known within the art may be used for the production of antibodies to a mutant centrobin protein. For example, monoclonal antibodies that bind to specific antigens may be obtained via the methods described in, e.g., Kohler et al., Nature, 256:495 (1975); Current Protocols in Immunology, Coligan et al., eds. 1:2.5.12.6.7, John Wiley & Sons (1991); Monoclonal Antibodies, Hybridomas: A New Dimension in Biological Analyses, Kennett, McKearn, and Bechtol eds., Plenum Press (1980); and Antibodies: A Laboratory Manual, Harlow and Lane eds., Cold Spring Harbor Laboratory Press (1988). Antibody fragments may be derived from intact antibodies using any suitable standard technique such as proteolytic digestion, or optionally, by proteolytic digestion (for example, using papain or pepsin) followed by mild reduction of disulfide bonds and alkylation. Alternatively, such fragments may also be generated by recombinant genetic engineering techniques, such as those techniques known in the art.

In certain aspects, the mutant centrobin is identified by detecting changes in function or activity compared to wild-type centrobin. In this regard, impaired binding to lamin A/C, reduced ability to mediate organized nuclear envelopes, misshapen and herniated nuclei, reduced localization to the nucleus, and/or regions of nuclear envelope pile-up suggest the presence of mutant centrobin. Methods of detecting binding activity include, for example, competitive binding assays; quantitative binding assays using instruments such as, for example, a Biacore® 3000 instrument; and chromatographic assays, e.g., HPLC and TLC.

### Screening Assays for Nuclear Envelope Modulators

The invention additionally features assays, including animal-based and cell-based assays, for identifying agents that modulate the integrity of nuclear lamina. For example, the invention provides a method of identifying an agent that enhances nuclear envelope integrity. The method comprises (a) administering an agent to a cell comprising mutant centrobin, (b) allowing the cell to replicate to form daughter cells, and (c) observing nuclear envelope morphology within the daughter cells. In one aspect, the cell is a fibroblast cell or a myoblast cell. A reduction in nuclear envelop defects in the daughter cell compared to the parent identifies an agent that enhances nuclear envelope integrity. In one embodiment, a cell producing mutant centrobin is incubated in the presence of an agent (i.e., test compound) and allowed to replicate. Alternatively, an agent is administered to an animal model of laminopathy and cells (e.g., striated muscle cells) are later collected to examine nuclear envelope morphology. Administration and dosing considerations applicable to animal models are described above. The mutant centrobin can comprise one or more of the centrobin mutations identified herein, or other mutations that result in nuclear envelope defects.

Once cellular replication has occurred in the presence of the agent, the nuclear envelope morphology of daughter cells is observed to determine if the laminar defects associated with mutant centrobin are corrected. The nuclear envelope is composed of the nuclear membrane, nuclear pore complexes, and the nuclear lamina. The nuclear lamina is located underneath the inner nuclear membrane and comprises a web of filaments, such as lamin family type V intermediate filament proteins (see, e.g., Boesin et al., Physiol. Rev., 86:967-1109 (2006)). The lattice structure of interwoven filaments imparts durability and stability to the lamina structure, which, in turn, supports the nuclear envelope and overall nuclear shape. Cells expressing mutant, non-functional centrobin display a highly unorganized nuclear envelope. The nuclear envelope can resemble a honeycomb, likely due to excess nuclear envelope accumulation at localized regions of the nucleus. The nuclear envelope defects lead to grossly misshapen, herniated nuclei. Thus, nuclear envelope morphology may be examined in several ways using any of a number of imaging techniques, such as electron microscopy. For example, the nuclear lamina of daughter cells can be viewed to detect enhanced interfilament organization (i.e., exposure to the agent promotes a more uniform laminar “lattice” structure compared to that of the afflicted parent cell). Alternatively or in addition, the nuclear envelope can be examined for increased organization (i.e., the envelope is less “honeycombed” compared to afflicted cells not exposed to the agent). Increased uniformity of the overall shape of the nucleus also points to a reduction in nuclear envelope defects and an increase in integrity. Exemplary imaging techniques are described in, for example, Aebi et al., Nature, 323:560-564 (1986); Gerace et al., J. Cell Sci Suppl., 1:137-60 (1984), and the Examples. With respect to an in vivo method for identifying an agent that enhances nuclear envelope integrity, amelioration or inhibition of a laminopathy in the animal model also signals that an agent enhances nuclear envelope integrity.

In addition, the invention provides a method of identifying an agent that restores the interaction of mutated centrobin and lamin or centrobin (optionally mutated) and mutated lamin, such as lamin comprising mutations in the tail domain. The method comprises (a) contacting mutated lamin A and centrobin with a candidate agent, (b) detecting binding of lamin A to centrobin. In one aspect, lamin A and centrobin are labeled with detection moieties, such as GST or His tag. Restoration of binding between lamin and centrobin can be detected using any suitable technique. For example, the binding complexes can be transferred to membrane and exposed to labeled anti-centrobin antibody and/or labeled anti-lamin antibody. Exemplary methods for detecting binding are described, e.g., in the Examples.

### Kits

The invention further provides a kit useful in the methods of the invention, such as a kit for diagnosing a laminopathy or identifying a predisposition to a laminopathy. The inventive kit comprises components useful in any of the methods described herein, including for example, nucleic acid primers, hybridization probes, restriction enzymes, binding agents that detect mutant centrobin, reagents for nucleic acid amplification, reagents for analyzing nucleic acid or amino acid sequences, and the like, as well as instructions for detecting mutant centrobin. For example, the kit comprises a detection agent selected from the group consisting of nucleic acid primers suitable for amplifying a centrobin coding sequence that facilitates detection of a centrobin mutation, nucleic acid probe(s) specific for a mutant centrobin coding sequence, and an antibody or fragment thereof that selectively binds mutant centrobin. The invention is not limited to a particular centrobin mutant, and encompasses detection of a truncated centrobin (e.g., an N-terminal fragment of wild-type centrobin), mutant centrobin resulting from expression with a frameshift in exon 19 of wild-type centrobin, and other mutant centrobin peptides resulting from incorrect splicing during expression. The kit also may detect mutant centrobin comprising an amino acid residue that corresponds to position 180 of SEQ ID NO: 3, wherein the residue is not proline; an amino acid residue that corresponds to position 480 of SEQ ID NO: 3, wherein the residue is not histidine; an amino acid residue that corresponds to position 539 of SEQ ID NO: 3, wherein the residue is not asparagine; and/or an amino acid residue that corresponds to position 870 of SEQ ID NO: 3, wherein the residue is not arginine. In one aspect, the kit comprises nucleic acid primers that amplify a portion of a centrobin coding sequence comprising exon 19 or exon 1 or encoding amino acid position 180, amino acid position 480, amino acid position 539, or amino acid position 870 of SEQ ID NO: 3. The invention also includes a kit for identifying an agent that enhances nuclear envelope integrity comprising the reagents described herein.

In addition to detection agents and instructions, the kits can include necessary buffers, primers for amplifying nucleic acids, enzymes (e.g., horseradish peroxidase, alkaline phosphatase, etc.), enzyme substrates, wash reagents, and the like. The components of the kit can be present in separate containers or on solid supports. Additionally, kits can provide reagents for assays to be used in combination with the methods of the invention, e.g., reagents for use with other laminopathy diagnostic assays.

### EXAMPLES

The invention, thus generally described, will be understood more readily by reference to the following examples, which are provided by way of illustration and are not intended to limit the invention.

### Example 1

This Example demonstrates that knockdown of centrobin results in grossly misshapen and herniated nuclei with disorganized nuclear envelope, a cellular phenotype associated with EDMD.

Centrobin was depleted in HeLa cells and U2OS cells by transfecting cells with centrobin siRNA#1 comprising the nucleic acid sequence 5′-AGUGCCACACUGCAGCAAC-3′ (SEQ ID NO: 7) (further described in Zou et al., supra). The siRNA transfection was performed with Oligofectamine (Invitrogen) as per the manufacturer's instructions. The siRNA efficiently knocked down the level of centrobin protein produced in transfected cells as determined by Western blot. The cells were stained with Topro-3 and observed via fluorescence microscopy. The percentage of 300 cells with misshapen and herniated nuclei was determined. When HeLa cells were transfected with centrobin siRNA#1, a striking 79% of transfected cells exhibited an abnormal nuclear morphology. Similar nuclear morphology defects were also observed in centrobin siRNA-transfected U2OS cells.

Immunostaining of the centrobin-depleted cells using antibodies against the nuclear envelope markers lamin A/C, lamin B1, LAP2, nuclearporins, and emerin revealed a severely disorganized nuclear envelope. Some centrobin-depleted cells displayed the typical “honeycomb” structure observed in cells harboring LMNA, EMD or nesprin mutations (Eriksson et al., Nature, 423:293-298 (2003); De Sandre-Giovannoli et al., Science, 300:2055 (2003); Chen et al., Lancet, 362:440-445 (2003); Navarro et al., Hum. Mol. Genet., 13:2493-2503 (2004); Muchir et al., Muscle Nerve, 30:444-450 (2004); Wang et al., Hum. Mol. Genet., 15:2479-2489 (2006); Vigouroux et al., J. Cell. Sci., 114:4459-4468 (2001)). Ultrastructural examination of centrobin-depleted HeLa cells was conducted using a Philips 110 electron microscope to view transfected cells fixed with 4% paraformaldehyde and 0.5% glutaraldehyde and stained with OsO₄ and urea acetate. Ultrastructural examination revealed that the honeycomb structure observed under immunofluorescence microscope is likely due to excess nuclear envelope pileup at localized regions of the nucleus.

To confirm that the observed nuclear morphology defects did not result from off-target effects of the particular centrobin siRNA used, two psuper-retro vector based centrobin RNAi constructs were generated. The first construct encoded the centrobin siRNA#1 described above. The second construct encoded a different centrobin RNAi (“centrobin siRNA#2”) that targets a different region of centrobin—the 3′ untranslated region. Centrobin siRNA#2 comprises the nucleic acid sequence 5′-CCCUCUCUCCUCUUUGUUC-3′ (SEQ ID NO: 8) (further described in Zou et al., supra). The vectors were used to transfect HeLa cells, which were subsequently stained with anti-lamin A/C antibody and Topro-3. Centrobin siRNA#2 (targeting the 3′ untranslated region of centrobin) mediated nuclear morphology defects similar to those caused by centrobin siRNA#1. The percentage of cells displaying abnormal nuclear shape was similar for both siRNA#1 and siRNA#2. siRNA#2-induced nuclear morphology defects were significantly rescued upon transfection of a myc-centrobin expression construct, which is resistant to siRNA#2 because the expression construct lacks centrobin's 3′ untranslated region. These results establish that RNAi-induced nuclear defects were not caused by off-target effects of the RNA constructs.

The ability of centrobin depletion to affect the nuclear morphology of cells arrested in cell cycles also was examined. HeLa cells were transfected with control, scrambled siRNA comprising the nucleic acid sequence 5′-CAGUCGCGUUUGCGACUGG-3′ (SEQ ID NO: 9) and centrobin siRNA#1. Six hours later, a portion of the cells was treated with 2 mM mimocine or 16 mM hydroxyurea for 72 hours. Mimocine treatment arrests cells in G1 phase while hydroxyurea treatment arrests the cells in the S phase. The cells were fixed and examined by immunofluorescence using anti-lamin A/C antibody and Topro-3 DNA stain. Western blotting revealed that centrobin siRNA decreased the level of centrobin protein in cells arrested in G1 or S phases. Immunofluorescence analysis surprisingly showed that nuclear morphology defects caused by centrobin depletion were significantly reduced in cells arrested in the G1 phase and cells arrested in the S phase of the cell cycle. Thus, centrobin functions during nuclear envelope assembly. Once the nucleus is assembled, centrobin depletion no longer has any overt effect on nuclear morphology.

This Example establishes that centrobin functions during nuclear envelope assembly, and inhibition leads to grossly misshapen and herniated nuclei, a phenotype remarkably similar to that of fibroblast cells derived from Emery-Dreifuss muscular dystrophy patients harboring lamin A or emerin mutations. The centrobin RNAi-induced nuclear morphology defects were the specific effects of centrobin depletion given that (1) two siRNA's targeting different regions of centrobin induced the similar nuclear morphology defects and (2) siRNA-induced defects were rescued by a centrobin expression construct resistant to siRNA.

### Example 2

This Example demonstrates the direct association of centrobin with lamin A, a nuclear envelope protein.

The ability of centrobin to directly bind lamins was determined using GST-laminA and His-centrobin fusion proteins in an overlay assay. Whole cell lysates were prepared from un-induced and induced BL21 cells harboring His-centrobin (comprising amino acids 1-903 of centrobin) and His-centrobin-aa365-903 (lacking the centrobin N-terminus). The BL21 cells were induced by addition of isopropylthiogalactoside (IPTG). The lysates were fractionated using SDS-PAGE, transferred to a PVDF membrane, probed with purified GST-laminA, and detected using anti-lamin A/C antibody. The same membrane was re-blotted using anti-His antibody to confirm the presence of centrobin peptide. The resulting blots showed that lamin A efficiently binds to full-length centrobin but not to the C-terminal fragment of centrobin (amino acids 365-903), indicating that lamin A binds to the N-terminal region of centrobin directly.

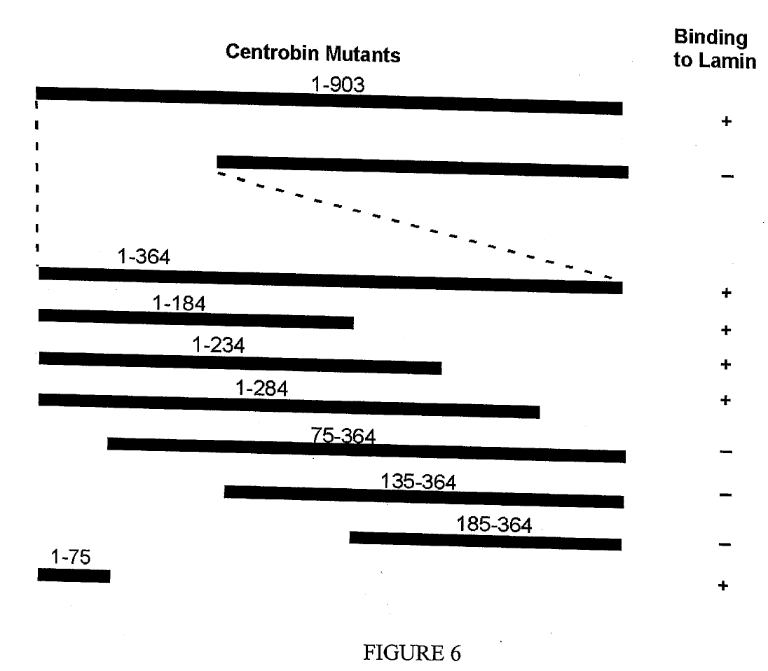

Further mutational analysis of the centrobin N-terminal region revealed that the N-terminal 75 amino acids of centrobin harbors the lamin A binding domain (FIG. 6). A Western blot was prepared using various portions of the N-terminal 364 amino acids of centrobin linked to a His tag. After fractionation and transference to a PVDF membrane, the PVDF membrane was probed using GST-lamin A. Centrobin-lamin A binding was detected using anti-lamin A/C antibody. The Western blot demonstrated that lamin A did not bind to centrobin mutants lacking the first 75 amino acids.

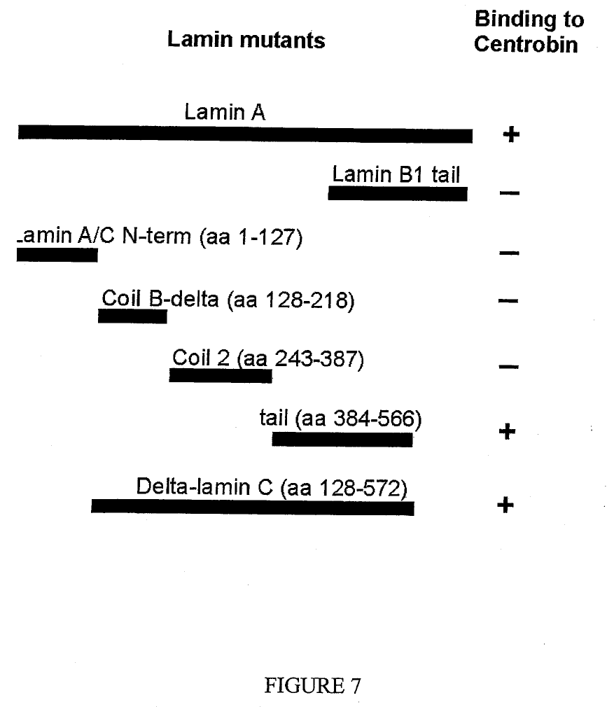

Using a set of lamin A/C mutants (described in Libotte et al., Mol. Biol. Cell, 16:3411-3424 (2005)), it was also determined that centrobin binds to the tail region of lamin A/C (amino acids 384-566) (FIG. 7). Purified GST-lamin A/C comprising the full length lamin sequence or truncated lamin sequences were fractionated using SDS-PAGE, transferred to a PVDF membrane, probed with His-myc-centrobin, and detected using anti-myc antibody. The same membrane was probed with anti-lamin A or stained by Ponseau S to show lamin protein levels. Centrobin interacted with lamin peptides comprising amino acids 384-566 of the lamin sequence. The tail domain of lamin A/C is mutated in EDMD, familial partial lipodystrophy (FPLD), dilated cardiomyopathy (DCM), limb girdle muscular dystrophy (LGMD), and mandibuloacral dysplasia (MAD).

Western blotting also established that centrobin binds specifically to lamin A/C. When PVDF membranes containing the C-terminus of lamin A and the C-terminus of lamin B1 were probed with His-myc-centrobin and detected with anti-myc antibody, lamin B1 tail-centrobin interactions were undetectable under conditions that allowed detection of lamin A/C tail-centrobin interactions. Thus, centrobin binds specifically to lamin A/C.

The effect of lamin A/C mutation on centrobin-lamin interactions also was examined. An R453W substitution in lamin A/C has been shown to be a causative mutation underlying EDMD in some patients. GST-lamin A tail fusion peptides, each comprising one of the following centrobin mutations, were fractionated on SDS-PAGE: R453W, E578V, L530P, R527P, R482W, R482Q, R482L, and deletion of residues 607-656 in pre-lamin A (“A50,” also known as progerin, which is associated with Hutchinson-Gilford Progeria Syndrome). The deleted residues in the Δ50 mutant contain the second proteolytic cleavage site involved in pre-lamin A processing. The peptides were transferred to a PVDF membrane, probed with His-myc-centrobin, and detected using an anti-myc antibody. The disease-associated lamin A/C mutation (R453W) significantly reduced interaction of the protein with centrobin. Three other lamin A/C mutants, L530P, R482L and R482W, also exhibited reduced binding with centrobin, although to a lesser extent.

To elucidate the function of lamin/centrobin interaction, the effect of overexpression of centrobin mutants containing the lamin-binding domain on nuclear envelope organization was examined. A GFP-centrobin (aal-184) fusion protein comprising a nuclear localization signal (NLS) and a GFP-centrobin fusion lacking an NLS were overexpressed in HeLa cells. The nuclear localization signal was added to simulate the endogenous full-length centrobin protein, which has an active nuclear localization signal at the C-terminus. Overexpression of the GFP-centrobin aa1-184 fusion protein with NLS resulted in misshapen and herniated nuclei. Immunostaining using anti-lamin A/C antibody indicated that the nuclear envelope was also disorganized in the transfected cells as evident by the presence of the typical “honeycomb” structure. The observed nuclear morphology defects are very similar to what were observed in the centrobin depleted cells. This finding further confirms that centrobin is critical for the integrity of nuclear envelope.

This Example demonstrates that for those diseases characterized by a decreased interaction of lamin A and centrobin, suitable therapeutic peptides would include full length centrobin and fragments thereof comprising the N-terminal 75 amino acids of the centrobin peptide. For those diseases or disorders characterized by excessive interaction between centrobin and lamin A, suitable therapeutic peptides would include binding inhibitors, such as anti-centrobin antibodies, that interfere with the interaction.

### Example 3

This Example demonstrates that at least a portion of centrobin localizes to the nucleus with lamin A/C and stabilizes the nuclear lamina.

In addition to binding lamin A/C, a portion of cellular centrobin localizes to the nuclear matrix. Centrobin levels in 2 million purified centrosomes were compared with the centrobin content of 1 million cells by Western blotting. Centrosomes were purified from HeLa cells, lysed in 1×SB buffer, fractionated on SDS-PAGE gel alongside HeLa whole cell lysate, and probed with anti-centrobin antibody. A majority of centrobin was observed to localize outside the centrosomes.

The intracellular distribution of centrobin was determined by cellular fractionation. HeLa cells were incubated in hypotonic buffer (15 mM KCl, 10 mM HEPES) for 30 minutes on ice. The cells were homogenized using a loose pestle, then centrifuged at 800 rpm to separate nuclear and cytoplasmic fractions. The cytoplasmic fraction was further centrifuged at 100,000 g to separate cytosol supernatant from cytosol pellet fraction. The nuclear fraction was further homogenized in 200 mM NaCl using a tight pestle, then centrifuged at 15,000 g to separate the nuclear supernatant from the nuclear pellet fraction. Western blots were generated by running equivalent amounts of each fraction alongside whole cell lysates on SDS-PAGE and probing the blots with anti-centrobin antibody. The Western blot revealed that approximately half of centrobin resides in the insoluble cytoplasm fraction, while most of the remaining half is located in the insoluble nuclear pellet fraction. Using a well-established nuclear-matrix preparation protocol (Capco et al., Cell, 29(3):847-58 (1982)), it was determined that approximately 40% of centrobin is in the nuclear matrix fraction, a fraction that also contains lamin A/C and lamin B. Immunofluorescence using a previously characterized anti-centrobin antibody (Zou et al., J. Cell. Biol., 171(3):437-45 (2005)) further corroborated this finding. The anti-centrobin antibody decorated the evenly distributed nuclear focus structures in addition to the typical centrosome staining, signaling the presence of centrobin in the nuclear matrix. Introduction of centrobin siRNA significantly diminished both nuclear and centrosomal staining of centrobin, indicating that the nuclear staining of centrobin was specific. Given that a significant portion of lamins also localize within the nucleus (Goldman et al., Genes Dev., 16:533-547 (2002)), it is likely that centrobin and lamins co-localize inside the nucleus.

Lamin mutation R453W, shown to disrupt centrobin-lamin binding in Example 2, also prevented centrobin localization to the nucleus. Centrobin distribution was examined in fibroblast cells of patients harboring an R482L mutation or an R453W mutation in lamin A/C. Normal fibroblast cells and fibroblasts from an EDMD patient with lamin R249Q mutation (a mutation localized outside the centrobin binding region) were used as controls. The R453W mutation was associated with a significant decrease in the amount of centrobin protein localized in cell nuclei, while lamin mutations R249Q and R482L did not affect the nuclear localization of centrobin. These results further establish that centrobin and lamin A/C co-localize in the nucleus.

The importance of both centrobin and lamin A/C to nuclear lamina integrity was highlighted in a double-knockdown study. HeLa cells were transfected with (i) lamin A siRNA alone or (ii) lamin A siRNA and centrobin siRNA#1. The nucleic acid sequence of the lamin A siRNA is 5′CUGGACUUCCAGAAGAACA-3′ (SEQ ID NO: 10) (available from Dharmacon, Inc., Lafayette, Colo.). The cells were stained with anti-lamin A/C and Topro-3 and examined under immunofluorescence. When transfected with lamin A siRNA alone, only very mild nuclear envelope disruption was observed, much milder than the phenotype induced by centrobin depletion. Depletion of both lamin A and centrobin surprisingly produced a much more pronounced mutant phenotype (i.e., a more disorganized nuclear envelope structure) than seen in cells transfected with centrobin siRNA alone or lamin A siRNA alone.

Western blotting analysis indicated that centrobin knockdown did not significantly change the total protein level of lamin A/C and lamin B. Similarly, lamin A/C knockdown did not significantly change the total protein level of centrobin. However, when the lamin A/C knockdown cells were fractionated into cytoplasm and nuclear fractions, the portion of centrobin in the nuclear fraction was decreased significantly in lamin A/C knockdown cells. More centrobin was observed in the nucleus than in the cytoplasm in control HeLa cells, while more centrobin was observed in the cytoplasm than in the nucleus in lamin A/C depleted cells. Similarly, in 293T cells, less centrobin was detected in the nucleus than in the cytoplasm, likely due to the extremely low level of lamin A/C. However, there was still less centrobin in the nucleus than in the cytoplasm even when 293T cells were reconstituted to express lamin A/C, indicating the reconstituted lamin A/C is not functional in 293T cells. These results indicate that lamin A/C interacts with centrobin physically and functionally and is essential for the integrity of the normal nuclear structure.

These findings evidence that centrobin localizes to the nucleus and, with lamin A/C, stabilizes nuclear laminar structure. Mutations in lamin A/C that cause Emery-Dreifuss muscular dystrophy diminished its binding to centrobin and led to a decrease of the amount of centrobin in the nucleus, suggesting that lamin A/C-centrobin binding is important to the nuclear localization of centrobin. In addition, double knockdown of centrobin and lamin A led to a more pronounced mutant nucleus phenotype than knockdown of centrobin or lamin A/C alone. These results suggest that agents that modulate the interaction and nuclear localization of centrobin and lamin can modulate nuclear envelope integrity and, by extension, laminopathy progression.

### Example 4

This Example demonstrates a correlation between EDMD and centrobin mutation.

Centrobin was sequenced from 59 probands with typical EDMD phenotypes that do not harbor LMNA, EMD, and nesprin mutations. Centrobin is encoded by 19 exons. Exon 1, encoding the lamin binding domain, was sequenced first. One deleterious centrobin mutation was identified in one of the EDMD probands. The identified exon 1 mutation is a heterozygous single nucleotide deletion (75delT) that leads to frame shift and translation stop (L27fs) in SEQ ID NO: 2. The patient is a typical female EDMD patient with contractures, skeletal muscular weakness, and severe heart involvement. The patient's son also suffers from EDMD and harbors the same exon 1 mutation (which is not present in the husband/father). The 75delT exon 1 mutation was not present in 340 alleles of an ethnically matched reference population, strongly indicating that the centrobin mutation is involved in EDMD pathogenesis. An additional 76 probands representing patients with EDMD symptoms was examined, leading to identification of seven additional centrobin exonic variants and one intronic variant associated with the disease. The identified mutations in the coding sequence of centrobin α (SEQ ID NO: 2) are summarized in Table 1.

TABLE 1

| | | | Number of |
|---|---|---|---|
| EXON/ | | Amino | patients |
| INTRON | cDNA orf | acids | affected |
| EXON 1 | C. 75delG | L27fs | 1 |
| EXON 4 | C. 539C > A | P180H | 1 |
| EXON 5 | C. 689C > T | T230I | 1 |
| EXON 10 | C. 1439A > T | H480L | 9 |
| EXON 12 | C. 1615A > T | N539Y | 12 |
| INTRON 16 | C. 1381-12G > A | | 3 |
| EXON 17 | C. 2512G > A | G838R | 1 |
| EXON 19 | C. 2699DelG | G900fs | 1 |
| EXON 19 | C. 2608 C > T | R870S | 1 |

The mutations identified herein affect 27 of 135 probands examined (20%). All the mutations are heterozygous. Three centrobin missense variants (T2301, G838R and R870S) were identified in EDMD probands and were not present in more than 380 controls of an ethically matched reference population. T2301 did not co-segregate with the disease. H480L and N539Y were the most common mutations identified in the EDMD probands tested, affecting 9 and 12 probands respectively. No homozygous mutations were identified for H480L and N539Y. All exonic mutants were predicted to be potentially damaging mutations using “polyPhen” (described in, e.g., Sunyaev et al., Human Molecular Genetics, 10(6):591-597 (2001); Sunyaev et al., Trends Genet, 16:198-200 (2000)). The intronic mutation, which is 12 nucleotides before the start of exon 17, was predicted to be part of an exonic splicing enhancer using “ESEfinder” (described in, e.g., Cartegni et al., Nucleic Acids Research, 31:3568-3571 (2003)).

Lymphocytes derived from the patient harboring the 75delT mutation were analyzed by Western blotting using anti-centrobin antibody. Jurkat cells, HL-60 cells, and two normal lymphocyte strains (one from a female donor and one from a male donor) were used as controls. Centrobin protein levels in the lymphocytes derived from the patient were much lower than that of control cells. The 75delT mutation leads to a frame shift and translation stop (L27fs). The mutated allele is predicted to encode a protein of 33 amino acids. Thus, cells harboring the centrobin 75delT mutation express a lower level of full-length centrobin.

Lymphocytes from the patient and lymphocytes from two controls also were stained with anti-lamin A/C, anti-emerin, and anti-nuclearporin antibodies. The cells were co-stained with Topro-3 for DNA. While control cells exhibited a normal nuclear shape and nuclear envelope, lymphocytes from the patient harboring the 75delT mutation exhibited abnormal nuclear shape and disorganized nuclear envelope, which was similar to the morphology observed in centrobin knockdown cells. Lymphocytes comprising centrobin mutations exhibited a convoluted, lobular nuclear appearance in contrast to the normal ovoid morphology seen in the majority of control cells. Heterogeneous Topro-3 staining of nuclear lobules was observed in cells with the centrobin mutation, suggesting that the nuclear shape abnormalities were accompanied by chromatin reorganization.

Paraffin-embedded, formalin-fixed muscle biopsies obtained from the patient were stained with anti-lamin A/C and anti-emerin antibodies. The muscle cells displayed an abnormal morphology with disorganized nuclear envelope, while control cells exhibited normal ovoid morphology and a normal nuclear envelope. In addition, the muscle cell nuclei in affected tissues were significantly elongated, and multiple nuclei were observed to be adjacent or “connected” to each other. In contrast, distinct nuclei of normal shape and size, equally spaced along the cells, were observed in control muscle cells.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed:

1. A method of treating laminopathy in a subject comprising administering a therapeutically effective amount of a centrobin polypeptide to the subject such that laminopathy is treated.

2. The method of claim 1, wherein the laminopathy is a laminopathic lipodystrophy disorder, a systemic laminopathy, a laminopathic neurological disorder, or a muscle laminopathy.

3. The method of claim 2, wherein the muscle laminopathy is Emery-Dreifuss muscular dystrophy, limb-girdle muscular dystrophy type 1B, congenital muscular dystrophy, multisystem dystrophy syndrome, dilated cardiomyopathy 1A, or dilated cardiomyopathy with conduction system defects.

4. The method of claim 3, wherein the laminopathy is Emery-Dreifuss muscular dystrophy type 2.

5. The method of claim 4, wherein administration of the centrobin polypeptide improves muscle strength, reduces muscle wasting, or reduces cardiomyopathy.

6. The method of claim 1, wherein the centrobin polypeptide is expressed from a nucleic acid molecule administered to the subject.

7. The method of claim 1, wherein the centrobin polypeptide is administered to the subject via direct injection into bone marrow or direct injection into muscle.

8. A method of identifying an agent that enhances nuclear envelope integrity, the method comprising (a) administering an agent to a cell comprising mutant centrobin, (b) allowing the cell to replicate to form daughter cells, and (c) observing nuclear envelope morphology within the daughter cells, wherein a reduction in nuclear envelope morphology defects in the daughter cells identifies an agent that enhances nuclear envelope integrity.

9. The method of claim 8, wherein the mutant centrobin is an N-terminal fragment of wild-type centrobin.

10. The method of claim 8, wherein the mutant centrobin comprises
(a) an amino acid residue that corresponds to position 180 of SEQ ID NO: 3, wherein the residue is not proline;
(b) an amino acid residue that corresponds to position 480 of SEQ ID NO: 3, wherein the residue is not histidine,
(c) an amino acid residue that corresponds to position 539 of SEQ ID NO: 3, wherein the residue is not asparagine; or
(d) an amino acid residue that corresponds to position 870 of SEQ ID NO: 3, wherein the residue is not arginine.

11. The method of claim 8, wherein the mutant centrobin results from expression comprising a frameshift in a location corresponding to exon 19 of wild-type centrobin.

12. The method of claim 8, wherein the mutant centrobin results from incorrect splicing during expression relative to wild-type splicing of centrobin.

13. A method for diagnosing or identifying a predisposition to a laminopathy, the method comprising detecting the presence or absence of mutant centrobin in a sample from a subject, wherein the presence of mutant centrobin indicates that the subject is suffering from, or is predisposed to develop, a laminopathy.

14. The method of claim 13, wherein the laminopathy is a muscle laminopathy.

15. The method of claim 14, wherein the muscle laminopathy is Emery-Dreifuss muscular dystrophy type 2.

16. The method of claim 13, wherein the method comprises detecting an N-terminal fragment of wild-type centrobin.

17. The method of claim 13, wherein the method comprises detecting a non-wild-type amino acid corresponding to a position in SEQ ID NO: 3 selected from the group consisting of position 180, position 480, position 539, and position 870.

18. The method of claim 13, wherein the method comprises detecting a frameshift in a location corresponding to exon 19 of wild-type centrobin.

19. A kit for identifying a predisposition to, or diagnosing, a laminopathy comprising (a) a detection agent selected from the group consisting of
(i) nucleic acid primers suitable for amplifying a centrobin coding sequence that facilitates detection of a mutation,
(ii) a nucleic acid probe specific for a mutant centrobin coding sequence, and
(iii) an antibody or fragment thereof that selectively binds mutant centrobin; and (b) instructions for detecting mutant centrobin.

20. The kit of claim 19, wherein the nucleic acid primers amplify a portion of a centrobin coding sequence corresponding to a sequence comprising exon 19 or exon 1 or encoding amino acid position 180, amino acid position 480, amino acid position 539, or amino acid position 870 of SEQ ID NO: 3.
